(19) United States
(12) Patent Application Publication
(10) Pub. No.: US 2022/0218427 A1
(43) Pub. Date: Jul. 14, 2022

(54) MEDICAL TOOL CONTROL SYSTEM, CONTROLLER, AND NON-TRANSITORY COMPUTER READABLE STORAGE

(71) Applicant: Sony Group Corporation, Tokyo (JP)

(72) Inventor: Kei TOMATSU, Tokyo (JP)

(73) Assignee: Sony Group Corporation, Tokyo (JP)

(21) Appl. No.: 17/614,572

(22) PCT Filed: Jun. 8, 2020
(86) PCT No.: PCT/JP2020/022479

§ 371 Date: Nov. 29, 2021

(30) Foreign Application Priority Data
Jun. 27, 2019 (JP) 2019-120349

(51) Int. Cl. A61B 34/37; A61B 34/00; A61B 90/50

(52) U.S. Cl. CPC A61B 34/37; A61B 2034/2057; A61B 90/50; A61B 34/76

(57) ABSTRACT

A medical tool control system includes a medical operation manipulator that detachably holds a medical tool. The system also includes circuitry configured to receive an input signal from an external device, evaluate a content of the input signal so as to determine a change in operation mode from a first control mode to a second control mode of the medical operation manipulator, wherein the first control mode and the second control mode have differing degrees of autonomy, and in at least one of the first control mode and the second control mode, the circuitry generates a control signal to drive a movement of the medical operation manipulator.

### TECHNICAL FIELD

The present disclosure relates to a medical tool control system, a controller, a non-transitory computer readable storage.

### BACKGROUND ART

In a recently disclosed method in the medical field, a multi-joint arm (also referred to as a support arm) provided with various medical units at the leading end of an arm is used when various kinds of medical treatment are performed.

For example, PTL 1 discloses a medical manipulator capable of preventing contact with tissues in the vicinity when the medical manipulator is inserted into a body and used.

### CITATION LIST

### Patent Literature

PTL 1: JP 2016-501042 A

### SUMMARY

### Technical Problem

In a method disclosed in the medical field, a medical operation is performed by using what is called an autonomous medical operation system configured to sense the environment of an operative field through a sensor and determine and execute the next operation based on recognition of the environment. As recognized by the present inventors, when such a medical operation system is used, it is expected that not the entire medical operation is performed by a medical manipulator but manipulation as part of the medical operation is performed by a doctor due to technological and ethical concerns. For example, in the case of a medical operation system that can be autonomously driven and that can be operated manually, it is expected that the system needs to be able to appropriately perform switching between the two modes.

Thus, the present disclosure presents a medical tool control system, a controller, a non-transitory computer readable storage, a medical operation support system, a control device, and a control method that are capable of appropriately performing switching between an autonomous drive mode and a manual operation mode of a medical operation manipulator. As an example, the manual control mode could be described as a first control mode, and the autonomous drive mode could be described as a second control mode. However, the present disclosure also includes modes with degrees of autonomy that span a spectrum from fully autonomous to fully manual. Thus, while the present description often uses manual mode and autonomous drive mode as examples, it should be understood that the disclosure is not limited to a binary selection of modes, but rather a full spectrum of modes, where there is a difference in level of autonomy between the first and second modes. In the present disclosure, autonomous drive includes both of a case in which the environment of an operative field is recognized based on a sensing result and then the next operation is determined and executed, and a case (semi-autonomous or supervised autonomous) in which a user performs or assists part (such as environment recognition and operation determination) of a series of operation.

### Solution to Problem

According to one embodiment, a medical tool control system includes a medical operation manipulator that detachably holds a medical tool; and

circuitry configured to
receive an input signal from an external device (such as a server connected with a network established inside and outside a hospital, a PC used by a medical staff, a projector installed in a conference room of the hospital, or a sensor provided in the medical operation manipulator),
evaluate a content of the input signal so as to determine a change in operation mode from a first control mode to a second control mode of the medical operation manipulator, wherein the first control mode and the second control mode have differing degrees of autonomy, and

in at least one of the first control mode and the second control mode, the circuitry generates a control signal to drive a movement of the medical operation manipulator.

According to a second embodiment, a controller for a medical operation manipulator that detachable holds a medical tool is described, the controller including:

circuitry configured to
receive an input signal from an external device,
evaluate a content of the input signal to determine a change in operation mode from a first control mode to a second control mode of the medical operation manipulator, wherein the first control mode and the second control mode have differing degrees of autonomy, and

in at least one of the first control mode and the second control mode, the circuitry generates a control signal to drive a movement of the medical operation manipulator.

According to a third embodiment, a non-transitory computer readable storage is described that has instructions that when executed by a processor cause the processor to perform a method, the method including:
receiving an input signal from an external device;
evaluating with circuitry a content of the input signal and determining a change in operation mode from a first control mode to a second control mode of a medical operation manipulator, wherein the first control mode and the second control mode have differing degrees of autonomy, and

in at least one of the first control mode and the second control mode, the circuitry generates a control signal to drive a movement of the medical operation manipulator.

### BRIEF DESCRIPTION OF DRAWINGS

FIG. 1 is a diagram schematically illustrating the entire configuration of a medical operation room system.

FIG. 2 is a diagram illustrating exemplary display of an operation screen on a centralized operation panel.

FIG. 3 is a diagram illustrating an exemplary situation of a medical operation to which the medical operation room system is applied.

FIG. 4 is a block diagram illustrating exemplary functional configurations of a camera head and a CCU illustrated in FIG. 3.

FIG. 5 is a schematic view illustrating the appearance of a support arm device according to an embodiment of the present disclosure.

FIG. 6 is a diagram illustrating an exemplary configuration of a master-slave apparatus according to the embodiment of the present disclosure.

FIG. 7 is a diagram illustrating an exemplary configuration of a medical operation support system according to the embodiment of the present disclosure.

FIG. 8 is a diagram illustrating an exemplary configuration of a medical operation manipulator according to the embodiment of the present disclosure.

FIG. 9 is a block diagram illustrating an exemplary configuration of a control device according to the embodiment of the present disclosure.

FIG. 10 is a flowchart illustrating the process of first processing at a control unit of the control device according to the embodiment of the present disclosure.

FIG. 11 is a flowchart illustrating the process of second processing at the control unit of the control device according to the embodiment of the present disclosure.

FIG. 12 is a flowchart illustrating an exemplary process of switching processing when a medical instrument mounted on the medical operation manipulator is a camera in the second processing at the control unit of the control device according to the embodiment of the present disclosure.

FIG. 13 is a flowchart illustrating an exemplary process of switching processing when the medical instrument mounted on the medical operation manipulator is a scalpel in the second processing at the control unit of the control device according to the embodiment of the present disclosure.

FIG. 14 is a flowchart illustrating an exemplary process of switching processing when the medical instrument mounted on the medical operation manipulator is a needle in the second processing at the control unit of the control device according to the embodiment of the present disclosure.

FIG. 15 is a flowchart illustrating an exemplary process of switching processing when the medical instrument mounted on the medical operation manipulator is a retractor in the second processing at the control unit of the control device according to the embodiment of the present disclosure.

FIG. 16 is a flowchart illustrating the process of third processing at the control unit of the control device according to the embodiment of the present disclosure.

FIG. 17 is a flowchart illustrating the process of fourth processing at the control unit of the control device according to the embodiment of the present disclosure.

FIG. 18 is a flowchart illustrating the process of fifth processing at the control unit of the control device according to the embodiment of the present disclosure.

FIG. 19 is a flowchart illustrating the process of sixth processing at the control unit of the control device according to the embodiment of the present disclosure.

FIG. 20 is a flowchart illustrating the process of seventh processing at the control unit of the control device according to the embodiment of the present disclosure.

FIG. 21 is a flowchart illustrating the process of eighth processing at the control unit of the control device according to the embodiment of the present disclosure.

FIG. 22 is a flowchart illustrating the process of ninth processing at the control unit of the control device according to the embodiment of the present disclosure.

FIG. 23 is a hardware configuration diagram illustrating an exemplary computer configured to achieve functions of an information processing device.

FIG. 24 is a block diagram of a computer that implements an artificial intelligence (AI) control engine according to an embodiment.

FIG. 25 is block diagram of a trained data extraction network according to an embodiment.

FIG. 26. is a block diagram of a data analysis network according to an embodiment.

FIG. 27 is a diagram of a concatenated source feature map according to an embodiment.

### DESCRIPTION OF EMBODIMENTS

Embodiments of the present disclosure are described in detail below with reference to the accompanying drawings. Sites identical to each other in the embodiments below are denoted by an identical reference sign, and duplicate description thereof is omitted.

The present disclosure will be described in accordance with the order of contents described below.

1. Exemplary configuration of medical operation system

2. Exemplary configuration of support arm device

3. Medical operation support system

3-1. Configuration of medical operation support system

3-2. Medical operation manipulator

3-3. Control device

4. Processing at control device

4-1. First processing

4-2. Second processing

4-2-1. Processing in a case of camera

4-2-2. Processing in a case of scalpel

4-2-3. Processing in a case of needle holder

4-2-4. Processing in a case of retractor

4-3. Third processing

4-4. Fourth processing

4-5. Fifth processing

4-6. Sixth processing

4-7. Seventh processing

4-8. Eighth processing

4-9. Ninth processing

5. Hardware configuration

### 1. Exemplary Configuration of Medical Operation System

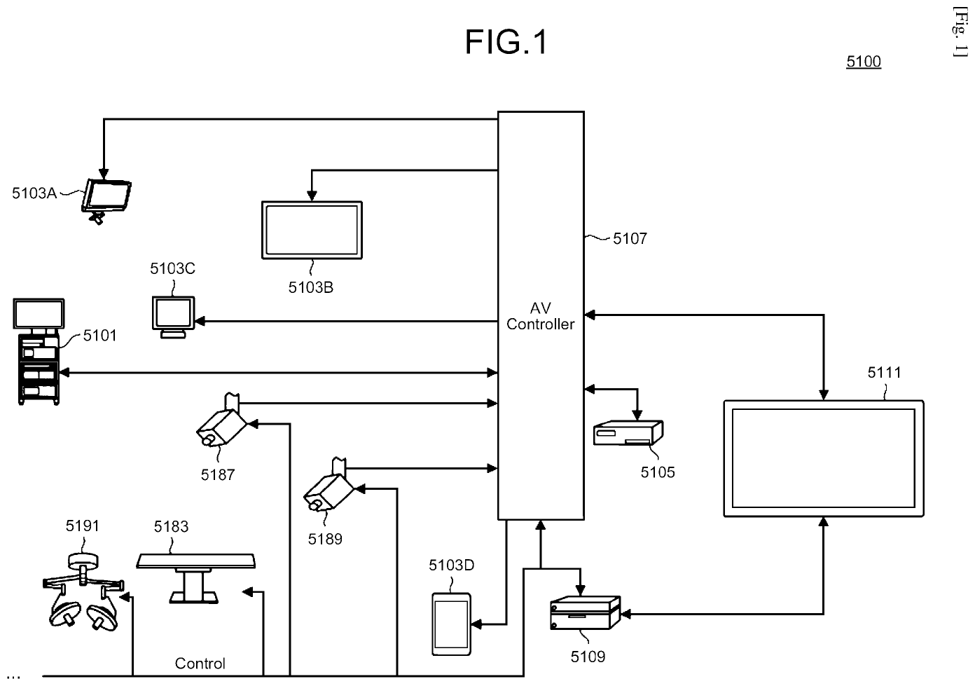

FIG. 1 is a diagram schematically illustrating the entire configuration of a medical operation room system 5100 to which a technology according to the present disclosure is applicable. As illustrated in FIG. 1, the medical operation room system 5100 has a configuration in which a group of devices installed in a medical operation room are cooperatively connected with each other through an audio-visual controller (AV controller) 5107 and a medical operation room control device 5109.

Various devices may be installed in the medical operation room. FIG. 1 illustrates, as examples, a group of various devices 5101 for endoscopic operations, a ceiling camera 5187 provided on the ceiling of the medical operation room and configured to capture an image of the vicinity of the hands of an operator, an operation place camera 5189 provided on the ceiling of the medical operation room and configured to capture an image of the situation of the entire medical operation room, a plurality of display devices 5103A to 5103D, a recorder 5105, a patient bed 5183, and an illumination 5191.

Among these devices, the group of devices 5101 belong to an endoscope medical operation system 5113 to be described later and include, for example, an endoscope and a display device configured to display an image captured by the endoscope. These devices belonging to the endoscope medical operation system 5113 are each also referred to as a medical instrument. The display devices 5103A to 5103D, the recorder 5105, the patient bed 5183, and the illumination 5191 are provided, for example, in the medical operation room, separately from the endoscope medical operation system 5113. These devices not belonging to the endoscope medical operation system 5113 are each also referred to as a non-medical instrument. The audio-visual controller 5107 and/or the medical operation room control device 5109 cooperatively control operation of the medical instruments and the non-medical instruments.

The audio-visual controller 5107 collectively controls processing related to image display at the medical and non-medical instruments. Specifically, among devices included in the medical operation room system 5100, the group of devices 5101, the ceiling camera 5187, and the operation place camera 5189 may be each a device (hereinafter also referred to as a transmission source device) having a function to transmit information (hereinafter also referred to as the display information) needed to be displayed in a medical operation. The display devices 5103A to 5103D may be each a device (hereinafter also referred to as an output destination device) to which the display information is output. The recorder 5105 may be a device corresponding to both the transmission source device and the output destination device. The audio-visual controller 5107 has a function to control operation of the transmission source device and the output destination device, acquire the display information from the transmission source device, and transmit the display information to the output destination device so that the display information is displayed or recorded. The display information includes various images captured in the medical operation, and various kinds of information (for example, body information and past examination results of a patient, and information on an operative method) related to the medical operation.

Specifically, information on an image of an operation site in the body cavity of the patient, which is captured by an endoscope, can be transmitted as the display information from the group of devices 5101 to the audio-visual controller 5107. In addition, information on an image of the vicinity of the hands of the operator, which is captured by the ceiling camera 5187 can be transmitted as the display information from the ceiling camera 5187. In addition, information on an image of the situation of the entire medical operation room, which is captured by the operation place camera 5189 can be transmitted as the display information from the operation place camera 5189. When the medical operation room system 5100 includes another device having an image capturing function, the audio-visual controller 5107 may acquire, as the display information from the other device, information on an image captured by the other device.

Alternatively, for example, information on these images captured in the past is recorded in the recorder 5105 by the audio-visual controller 5107. The audio-visual controller 5107 can acquire, as the display information from the recorder 5105, information on the images captured in the past. In addition, various kinds of information related to the medical operation may be recorded in the recorder 5105 in advance.

The audio-visual controller 5107 causes at least one of the display devices 5103A to 5103D as the output destination device to display the acquired display information (in other words, an image captured in the medical operation and various kinds of information related to the medical operation). In the illustrated example, the display device 5103A is a device installed being suspended from the ceiling of the medical operation room, the display device 5103B is a device installed on the wall surface of the medical operation room, the display device 5103C is a device installed on a desk in the medical operation room, and the display device 5103D is a mobile device (for example, a tablet personal computer (PC)) having a display function.

Although not illustrated in FIG. 1, the medical operation room system 5100 may include a device outside the medical operation room. The device outside the medical operation room is, for example, a server connected with a network established inside and outside a hospital, a PC used by a medical staff, or a projector installed in a conference room of the hospital. When such an external device is disposed outside the hospital, the audio-visual controller 5107 may cause a display device at another hospital to display the display information through a video conference system and the like for remote medical care.

The medical operation room control device 5109 collectively controls processing other than processing related to image display at a non-medical instrument. For example, the medical operation room control device 5109 controls drive of the patient bed 5183, the ceiling camera 5187, the operation place camera 5189, and the illumination 5191.

The medical operation room system 5100 includes a centralized operation panel 5111 through which a user can provide an instruction on image display to the audio-visual controller 5107 and provide an instruction on operation of a non-medical instrument to the medical operation room control device 5109. In the centralized operation panel 5111, a touch panel is provided on the display surface of a display device.

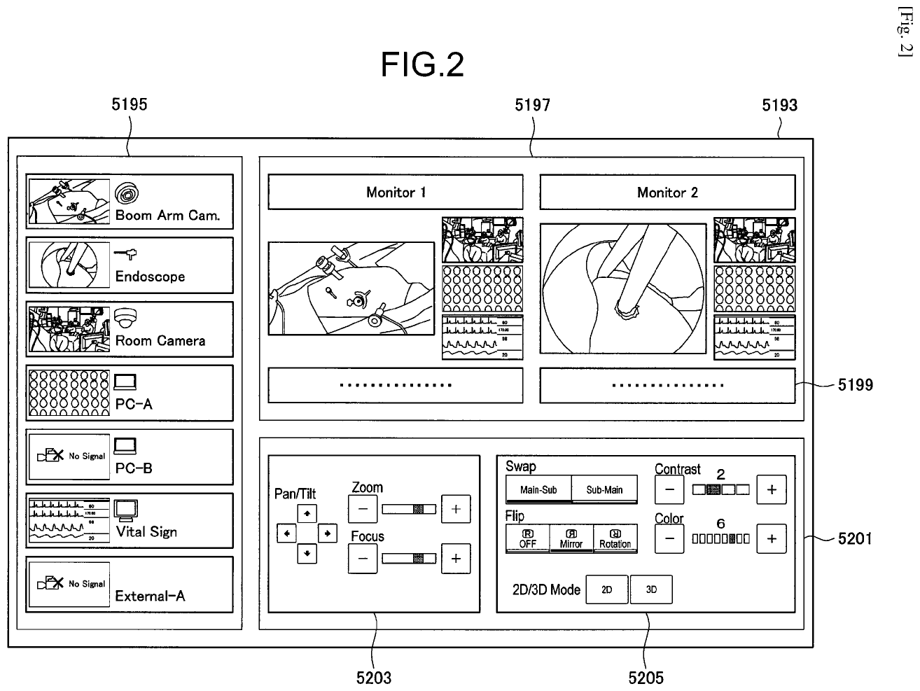

FIG. 2 is a diagram illustrating exemplary display of an operation screen on the centralized operation panel 5111. FIG. 2 illustrates, as an example, an operation screen corresponding to a case in which the medical operation room system 5100 is provided with two display devices as the output destination devices. As illustrated in FIG. 2, this operation screen 5193 includes a transmission source selection region 5195, a preview region 5197, and a control region 5201.

The transmission source selection region 5195 displays each transmission source device included in the medical operation room system 5100, and a thumbnail screen of the display information held at the transmission source device in association with each other. The user can select the display information to be displayed on a display device from among the transmission source devices displayed in the transmission source selection region 5195.

The preview region 5197 displays previews of screens displayed on the two display devices (Monitor 1 and Monitor 2) as the output destination devices. In the illustrated example, four images are displayed in a Picture-in-Picture scheme for each display device. The four images correspond to the display information transmitted from a transmission source device selected in the transmission source selection region 5195. One of the four images is displayed relatively large as a main image, and the remaining three images are displayed relatively small as sub images. The user can interchange the main image and the sub images by selecting regions in which the four images are displayed as appropriate. In addition, a status display region 5199 is provided at a part below the regions in which the four images are displayed, and a status (for example, the elapsed time of the medical operation, and body information of the patient) related to the medical operation can be displayed in this region as appropriate.

The control region 5201 includes a transmission source operation region 5203 in which a graphical user interface (GUI) component for performing an operation on each transmission source device is displayed, and an output destination operation region 5205 in which a GUI component for performing an operation on each output destination device is displayed. In the illustrated example, the transmission source operation region 5203 includes GUI components for performing various operations (panning, tilting, and zooming) to a camera in a transmission source device having an image capturing function. The user can operate the operation of the camera in the transmission source device by selecting the GUI components as appropriate. Although not illustrated, when the recorder is selected as a transmission source device in the transmission source selection region 5195 (in other words, when an image recorded in the recorder in the past is displayed in the preview region 5197), the transmission source operation region 5203 may include GUI components for performing operations such as playback, playback stop, rewind, and fast forward of this image.

The output destination operation region 5205 includes GUI components for performing various operations (swap, flip, color adjustment, contrast adjustment, switching between 2D display and 3D display) for display on each display device as the output destination device. The user can operate display on the display device by selecting these GUI components as appropriate.

The operation screen displayed on the centralized operation panel 5111 is not limited to the illustrated example, but the user may be able to input, through the centralized operation panel 5111, an operation to each device included in the medical operation room system 5100 and controlled by the audio-visual controller 5107 and the medical operation room control device 5109.

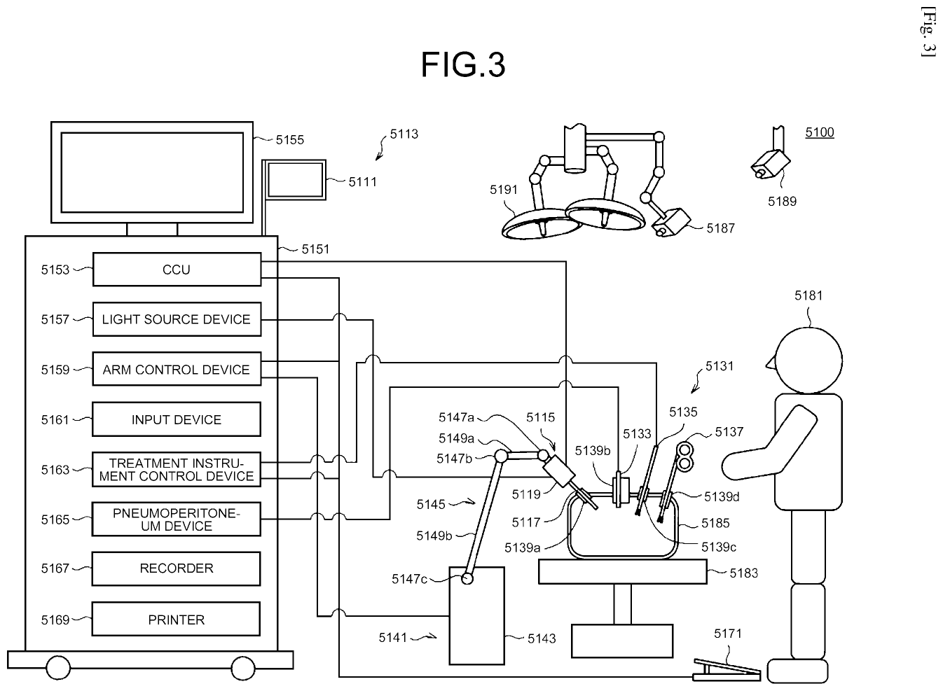

FIG. 3 is a diagram illustrating an exemplary situation of a medical operation to which the above-described medical operation room system is applied. The ceiling camera 5187 and the operation place camera 5189 are provided on the ceiling of the medical operation room and can capture images of the vicinity of the hands of an operator (doctor) 5181 performing treatment on an affected part of a patient 5185 on the patient bed 5183 and the situation of the entire medical operation room. The ceiling camera 5187 and the operation place camera 5189 may have, for example, a magnification adjustment function, a focal length adjustment function, and an image capturing direction adjustment function. The illumination 5191 is provided on the ceiling of the medical operation room and irradiates at least the vicinity of the hands of the operator 5181. The illumination 5191 may be able to adjust the quantity of irradiation light, the wavelength (color) of irradiation light, the direction of irradiation light, and the like as appropriate.

As illustrated in FIG. 1, the endoscope medical operation system 5113, the patient bed 5183, the ceiling camera 5187, the operation place camera 5189, and the illumination 5191 are cooperatively connected with each other through the audio-visual controller 5107 and the medical operation room control device 5109 (not illustrated in FIG. 3). The centralized operation panel 5111 is provided in the medical operation room, and the user can operate these devices in the medical operation room as appropriate through the centralized operation panel 5111 as described above.

The following describes the configuration of the endoscope medical operation system 5113 in detail. As illustrated in FIG. 3, the endoscope medical operation system 5113 includes an endoscope 5115, other operation instruments 5131, a support arm device 5141 supporting the endoscope 5115, and a cart 5151 on which various devices for an endoscopic operation are mounted.

In the endoscopic operation, instead of being cut to open the abdominal cavity, the abdominal wall is punctured with a plurality of tubular puncture instruments called trocars 5139a to 5139d. Then, a lens barrel 5117 of the endoscope 5115 and the other operation instruments 5131 are inserted into the body cavity of the patient 5185 through the trocars 5139a to 5139d. In the illustrated example, a pneumoperitoneum tube 5133, an energy treatment instrument 5135, and forceps 5137 are inserted into the body cavity of the patient 5185 as the other operation instruments 5131. The energy treatment instrument 5135 is a treatment instrument configured to perform tissue incision and peeling, blood vessel sealing, and the like with high frequency current or ultrasonic wave vibration. However, the illustrated operation instruments 5131 are merely exemplary and may be various operation instruments, such as a prick and a retractor, typically used in an endoscopic operation.

A display device 5155 displays an image of an operation site in the body cavity of the patient 5185, which is captured by the endoscope 5115. The operator 5181 performs treatment such as resection of an affected part by using the energy treatment instrument 5135 and the forceps 5137 while watching, in real time, the image of the operation site displayed on the display device 5155. Although not illustrated, the pneumoperitoneum tube 5133, the energy treatment instrument 5135, and the forceps 5137 are supported by the operator 5181, an assistant, or the like in a medical operation.

(Support Arm Device)

The support arm device 5141 includes an arm unit 5145 extending from a base unit 5143. In the illustrated example, the arm unit 5145 includes joint parts 5147a, 5147b, and 5147c and links 5149a and 5149b and is driven under control of an arm control device 5159. The arm unit 5145 supports the endoscope 5115 and controls the position and posture thereof. Accordingly, the position of the endoscope 5115 is stably fixed.

(Endoscope)

The endoscope 5115 includes the lens barrel 5117, a region of which extending from the leading end by a predetermined length is inserted into the body cavity of the patient 5185, and a camera head 5119 connected with the base end of the lens barrel 5117. In the illustrated example, the endoscope 5115 is configured as what is called a rigid scope including the lens barrel 5117 that is rigid, but the endoscope 5115 may be configured as what is called a flexible scope including the lens barrel 5117 that is flexible.

An opening part to which an objective lens is fitted is provided at the leading end of the lens barrel 5117. A light source device 5157 is connected with the endoscope 5115, and light generated by the light source device 5157 is guided to the leading end of the lens barrel by a light guide extending inside the lens barrel 5117 and is emitted toward an observation target in the body cavity of the patient 5185 through the objective lens. The endoscope 5115 may be a direct view scope, an angled view scope, or a side view scope.

An optical system and an image sensor are provided inside the camera head 5119, and reflected light (observation light) from the observation target is condensed onto the image sensor through the optical system. The image sensor performs photoelectric conversion of the observation light and generates an electric signal corresponding to the observation light, in other words, an image signal corresponding to an observation image. The image signal is transmitted to a camera control unit (CCU) 5153 as RAW data. The camera head 5119 has a function to adjust the magnification and the focal length by driving the optical system as appropriate.

The camera head 5119 may include a plurality of image sensors to support, for example, stereoscopic viewing (3D display). In this case, a plurality of relay optical systems are provided inside the lens barrel 5117 to guide the observation light to each image sensor.

(Various Devices Mounted on Cart)

The CCU 5153 is achieved by a central processing unit (CPU), a graphics processing unit (GPU), or the like and collectively controls the operation of the endoscope 5115 and the display device 5155. Specifically, the CCU 5153 performs, on the image signal received from the camera head 5119, various kinds of image processing, such as image development processing (de-mosaic processing), for displaying an image based on the image signal. The CCU 5153 provides the image signal subjected to the image processing to the display device 5155. The CCU 5153 is connected with the audio-visual controller 5107 illustrated in FIG. 1. The CCU 5153 also provides the image signal subjected to the image processing to the audio-visual controller 5107. In addition, the CCU 5153 transmits a control signal to the camera head 5119 to control drive thereof. The control signal may include information related to image capturing conditions, such as the magnification and the focal length. The information related to image capturing conditions may be input through an input device 5161 or through the centralized operation panel 5111 described above.

The display device 5155 displays, under control of the CCU 5153, the image based on the image signal subjected to the image processing by the CCU 5153. When the endoscope 5115 supports image capturing at a high resolution such as 4K (the number of horizontal pixels 3840×the number of vertical pixels 2160) or 8K (the number of horizontal pixels 7680×the number of vertical pixels 4320) and/or supports 3D display, the display device 5155 may be a display device capable of performing display at the high resolution and/or capable of performing 3D display, respectively. When the display device 5155 supports image capturing at a high resolution such as 4K or 8K, an enhanced sense of immersion can be obtained with the display device 5155 having a size of 55 inches or larger. A plurality of display devices 5155 between which the resolution and the size are different may be provided in accordance with usage.

The light source device 5157 is achieved by a light source such as a light emitting diode (LED) and supplies irradiation light for image capturing of an operation site to the endoscope 5115.

The arm control device 5159 is achieved by a processor such as a CPU and operates in accordance with a predetermined computer program to control drive of the arm unit 5145 of the support arm device 5141 in accordance with a predetermined control scheme.

The input device 5161 is an input interface for the endoscope medical operation system 5113. The user can input various kinds of information and instructions to the endoscope medical operation system 5113 through the input device 5161. For example, the user inputs, through the input device 5161, various kinds of information related to a medical operation such as body information of the patient and information on the operative method of the medical operation. In addition, for example, the user inputs, through the input device 5161, an instruction to drive the arm unit 5145, an instruction to change image capturing conditions (such as the kind of irradiation light, the magnification, and the focal length) for the endoscope 5115, an instruction to drive the energy treatment instrument 5135, and the like.

The kind of the input device 5161 is not limited, but the input device 5161 may be various kinds of well-known input devices. The input device 5161 may be, for example, a mouse, a keyboard, a touch panel, a switch, a foot switch 5171, and/or a lever. When the input device 5161 is a touch panel, the touch panel may be provided on the display surface of the display device 5155.

Alternatively, the input device 5161 is a device mounted on the user, such as a glasses-type wearable device or a head mounted display (HMD), and various kinds of inputting in accordance with the gesture and sight line of the user detected by the device are performed through the input device 5161. The input device 5161 includes a camera capable of detecting motion of the user, and various kinds of input in accordance with the gesture and sight line of the user detected from a video captured by the camera are performed through the input device 5161. The input device 5161 further includes a microphone capable of collecting voice of the user, and various kinds of inputting by the voice through the microphone are performed through the input device 5161. Since various kinds of information can be input through the input device 5161 in such a non-contact manner, the user (for example, the operator 5181) belonging to a clean area can operate an instrument belonging to an unclean area in a non-contact manner. In addition, the user can operate an operation instrument being held without releasing hand from the instrument, which leads to improvement of convenience for the user.

A treatment instrument control device 5163 controls drive of the energy treatment instrument 5135 for tissue cauterization, incision, blood vessel sealing, or the like. A pneumoperitoneum device 5165 feeds gas into the body cavity through the pneumoperitoneum tube 5133 so that the body cavity of the patient 5185 is inflated to obtain the visual field of the endoscope 5115 and a work space for the operator. A recorder 5167 is a device capable of recording various kinds of information related to the medical operation. A printer 5169 is a device capable of printing various kinds of information related to the medical operation in various formats of text, image, graph, and the like.

The following describes particularly characteristic components in the endoscope medical operation system 5113 in more detail.

(Support Arm Device)

The support arm device 5141 includes the base unit 5143 as a base, and the arm unit 5145 extending from the base unit 5143. In the illustrated example, the arm unit 5145 includes the joint parts 5147a, 5147b, and 5147c, and the links 5149a and 5149b coupled with each other through the joint part 5147b, but FIG. 3 illustrates the configuration of the arm unit 5145 in a simplified manner for simplification. In reality, for example, the shapes, numbers, and disposition of the joint parts 5147a to 5147c and the links 5149a and 5149b, and the directions of the rotational axes of the joint parts 5147a to 5147c may be set as appropriate so that the arm unit 5145 has a desired freedom. For example, the arm unit 5145 may be excellently configured to have six degrees of freedom or more. Accordingly, the endoscope 5115 can be freely moved in the movable range of the arm unit 5145, and thus the lens barrel 5117 of the endoscope 5115 can be inserted into the body cavity of the patient 5185 in a desired direction.

The joint parts 5147a to 5147c are each provided with an actuator and rotatable about a predetermined rotational axis through drive of the actuator. As drive of the actuator is controlled by the arm control device 5159, the rotation angle of each of the joint parts 5147a to 5147c is controlled, and drive of the arm unit 5145 is controlled. Accordingly, the position and posture of the endoscope 5115 are controlled. In this case, the arm control device 5159 can control drive of the arm unit 5145 in various well-known control schemes such as force control and position control.

For example, when the operator 5181 performs operation inputting through the input device 5161 (including the foot switch 5171) as appropriate, drive of the arm unit 5145 may be controlled by the arm control device 5159 in accordance with the operation input as appropriate to control the position and posture of the endoscope 5115. Through this control, the endoscope 5115 at the leading end of the arm unit 5145 can be moved from an optional position to another optional position, and then fixedly supported at the position after the movement. The arm unit 5145 may be operated in what is called a master-slave scheme. In this case, the arm unit 5145 may be remotely operated by the user through the input device 5161 installed at a place separated from the medical operation room.

When force control is applied, the arm control device 5159, what is called power assist control may be performed in which the actuators of the joint parts 5147a to 5147c are driven so that the arm unit 5145 receives external force from the user and is smoothly moved by the external force. Accordingly, when moving the arm unit 5145 while directly touching the arm unit 5145, the user can move the arm unit 5145 with relatively small force. Thus, the endoscope 5115 can be more intuitively moved through a simpler operation, which leads to improvement of convenience for the user.

Typically, in an endoscopic operation, the endoscope 5115 is supported by a doctor called a scopist. However, when the support arm device 5141 is used, the position of the endoscope 5115 can be more reliably fixed without a manual operation, and thus an image of an operation site can be reliably obtained, which allows smooth execution of a medical operation.

The arm control device 5159 does not necessarily need to be provided to the cart 5151. In addition, the number of arm control devices 5159 does not necessarily need to be one. For example, the arm control device 5159 may be provided to each of the joint parts 5147a to 5147c of the arm unit 5145 of the support arm device 5141, and drive control of the arm unit 5145 may be achieved through cooperation of a plurality of arm control devices 5159 with each other.

(Light Source Device)

The light source device 5157 supplies irradiation light for image capturing of an operation site to the endoscope 5115. The light source device 5157 is achieved by a white light source configured as, for example, an LED, a laser beam source, or a combination thereof. In this case, when the white light source is configured as a combination of RGB laser beam sources, the output intensity and output timing of each color (wavelength) can be highly accurately controlled, and thus the white balance of a captured image can be adjusted at the light source device 5157. In addition, in this case, an image corresponding to each of RGB can be captured in a time divisional manner by irradiating an observation target with laser beams from the respective RGB laser beam sources in a time divisional manner and controlling drive of the image sensors of the camera head 5119 in synchronization with the timings of irradiation. With this method, a color image can be obtained without providing color filters to the image sensors.

In addition, drive of the light source device 5157 may be controlled so that the intensity of output light is changed in every predetermined time. Drive of the image sensors of the camera head 5119 is controlled in synchronization with the timing of the light intensity change to acquire images in a time divisional manner. The images can be synthesized to generate a high dynamic range image without what are called underexposure and overexposure.

The light source device 5157 may be capable of supplying light in a predetermined wavelength band for special light observation. In the special light observation, for example, what is called narrow band light observation (narrow band imaging) is performed in which an image of a predetermined tissue such as a blood vessel on the surface layer of mucous membrane is captured at high contrast through irradiation with light in a band narrower than the band of irradiation light (in other words, white light) in normal observation by using the wavelength dependency of light absorption at a body tissue. Alternatively, in the special light observation, fluorescence observation may be performed in which an image is obtained by using fluorescence generated through irradiation with excitation light. In the fluorescence observation, for example, a body tissue is irradiated with excitation light to observe fluorescence from the body tissue (self-fluorescence observation), or a reagent such as indocyanine green (ICG) is locally injected into a body tissue and the body tissue is irradiated with excitation light corresponding to the fluorescence wavelength of the reagent to obtain a fluorescence image. The light source device 5157 may be capable of supplying the narrow band light and/or excitation light corresponding to such special light observation.

(Camera Head and CCU)

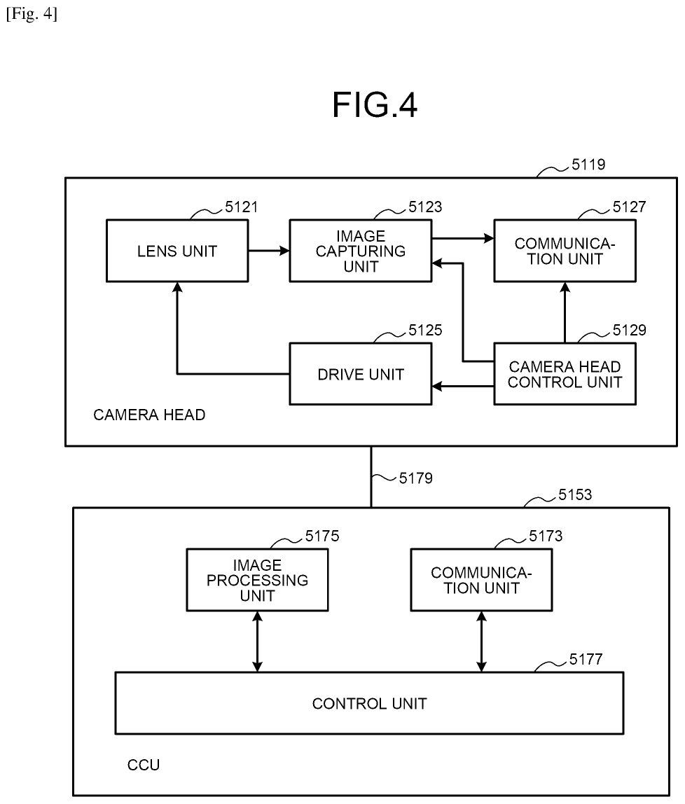

The following describes functions of the camera head 5119 and the CCU 5153 of the endoscope 5115 in more detail with reference to FIG. 4. FIG. 4 is a block diagram illustrating exemplary functional configurations of the camera head 5119 and the CCU 5153 illustrated in FIG. 3.

As illustrated in FIG. 4, the camera head 5119 includes, as functions thereof, a lens unit 5121, an image capturing unit 5123, a drive unit 5125, a communication unit 5127, and a camera head control unit 5129. The CCU 5153 includes, as functions thereof, a communication unit 5173, an image processing unit 5175, and a control unit 5177. The camera head 5119 and the CCU 5153 are connected with each other through a transmission cable 5179 to perform bidirectional communication therebetween.

The following first describes a functional configuration of the camera head 5119. The lens unit 5121 is an optical system provided at a connection part with the lens barrel 5117. The observation light acquired from the leading end of the lens barrel 5117 is guided to the camera head 5119 and incident on the lens unit 5121. The lens unit 5121 is formed by combining a plurality of lenses including a zoom lens and a focus lens. The optical property of the lens unit 5121 is adjusted so that the observation light is condensed onto the light-receiving surface of an image sensor of the image capturing unit 5123. The position of each of the zoom and focus lenses on the optical axis thereof is movable for adjustment of the magnification and focal point of a captured image.

The image capturing unit 5123 is achieved by an image sensor and disposed at a later stage of the lens unit 5121. The observation light having passed through the lens unit 5121 is condensed onto the light-receiving surface of the image sensor and subjected to photoelectric conversion through which an image signal corresponding to the observation image is generated. The image signal generated by the image capturing unit 5123 is provided to the communication unit 5127.

The image sensor as the image capturing unit 5123 is, for example, an image sensor of a complementary metal oxide semiconductor (CMOS) type, which includes a Bayer array and is capable of performing color image capturing. The image sensor may be, for example, an image sensor that can support capturing of an image at a high resolution of 4K or higher. When an image of an operation site is obtained at a high resolution, the operator 5181 can understand the situation of the operation site in more detail and perform a medical operation more smoothly.

The image sensor as the image capturing unit 5123 includes a pair of image sensors for acquiring right-eye and left-eye image signals for 3D display, respectively. When 3D display is performed, the operator 5181 can more accurately understand the depth of a biological tissue at an operation site. When the image capturing unit 5123 has a multiple-plate configuration, a plurality of lens units 5121 are provided for the respective image sensors.

The image capturing unit 5123 does not necessarily need to be provided to the camera head 5119. For example, the image capturing unit 5123 may be provided right after the objective lens inside the lens barrel 5117.

The drive unit 5125 is achieved by an actuator and moves each of the zoom and focus lenses of the lens unit 5121 along the optical axis by a predetermined distance under control of the camera head control unit 5129. Accordingly, the magnification and focal point of an image captured by the image capturing unit 5123 can be adjusted as appropriate.

The communication unit 5127 is achieved by a communication device for communicating various kinds of information with the CCU 5153. The communication unit 5127 transmits an image signal acquired from the image capturing unit 5123 to the CCU 5153 through the transmission cable 5179 as RAW data. The image signal is preferably transmitted through optical communication to display a captured image of an operation site at low latency. This is because, in a medical operation, the operator 5181 performs the medical operation while observing the state of an affected part based on a captured image, and thus a moving image of an operation site needs to be displayed in real time as much as possible to perform the medical operation in a safer and more reliable manner. When optical communication is performed, the communication unit 5127 is provided with a photoelectric conversion module configured to convert an electric signal into an optical signal. After converted into an optical signal by the photoelectric conversion module, the image signal is transmitted to the CCU 5153 through the transmission cable 5179.

The communication unit 5127 receives a control signal for controlling drive of the camera head 5119 from the CCU 5153. The control signal includes information related to image capturing conditions, such as information on specification of the frame rate of a captured image, information on specification of an exposure value at image capturing, and/or information on specification of the magnification and focal point of the captured image. The communication unit 5127 provides the received control signal to the camera head control unit 5129. The control signal from the CCU 5153 may be transmitted through optical communication. In this case, the communication unit 5127 is provided with a photoelectric conversion module configured to convert an optical signal into an electric signal. After converted into an electric signal by the photoelectric conversion module, the control signal is provided to the camera head control unit 5129.

The above-described image capturing conditions such as the frame rate, the exposure value, the magnification, and the focal point are automatically set by the control unit 5177 of the CCU 5153 based on the acquired image signal. Accordingly, the endoscope 5115 has what are called an auto exposure (AE) function, an auto focus (AF) function, and an auto white balance (AWB) function.

The camera head control unit 5129 controls drive of the camera head 5119 based on the control signal received from the CCU 5153 through the communication unit 5127. For example, the camera head control unit 5129 controls drive of the image sensor of the image capturing unit 5123 based on information on specification of the frame rate of a captured image and/or information on specification of exposure at image capturing. In addition, for example, the camera head control unit 5129 moves the zoom and focus lenses of the lens unit 5121 as appropriate through the drive unit 5125 based on information on specification of the magnification and focal point of the captured image. The camera head control unit 5129 may further have a function to store information for identifying the lens barrel 5117 and the camera head 5119.

The camera head 5119 can have resistance against autoclave sterilization treatment by disposing components such as the lens unit 5121 and the image capturing unit 5123 in a sealing structure having high air-tightness and water-tightness.

The following describes a functional configuration of the CCU 5153. The communication unit 5173 is achieved by a communication device for communicating various kinds of information with the camera head 5119. The communication unit 5173 receives an image signal transmitted from the camera head 5119 through the transmission cable 5179. The image signal may be excellently transmitted through optical communication as described above. In this case, to support optical communication, the communication unit 5173 is provided with a photoelectric conversion module configured to convert an optical signal into an electric signal. The communication unit 5173 provides the image signal converted into an electric signal to the image processing unit 5175.

In addition, the communication unit 5173 transmits a control signal for controlling drive of the camera head 5119 to the camera head 5119. The control signal may be transmitted through optical communication.

The image processing unit 5175 performs various kinds of image processing on an image signal as RAW data transmitted from the camera head 5119. Examples of the image processing include various kinds of well-known signal processing such as image development processing, high-image-quality processing (for example, band enhancement processing, super-resolution processing, noise reduction (NR) processing, and/or hand-shake correction processing), and/or enlargement processing (electronic zoom processing). In addition, the image processing unit 5175 performs detection processing on the image signal for performing AE, AF, and AWB.

The image processing unit 5175 is achieved by a processor such as a CPU or a GPU, and the above-described image processing and detection processing are performed when the processor operates in accordance with a predetermined computer program. When the image processing unit 5175 is achieved by a plurality of GPUs, the image processing unit 5175 divides information of the image signal as appropriate and performs image processing at the GPUs in parallel.

The control unit 5177 performs various kinds of control related to image capturing of an operation site by the endoscope 5115 and display of a captured image. For example, the control unit 5177 generates a control signal for controlling drive of the camera head 5119. In this case, when image capturing conditions are input by the user, the control unit 5177 generates the control signal based on the inputting by the user. Alternatively, when the endoscope 5115 has the AE function, the AF function, and the AWB function, the control unit 5177 calculates optimum exposure value, focal length, and white balance as appropriate in accordance with a result of the detection processing by the image processing unit 5175, and generates the control signal.

In addition, the control unit 5177 causes the display device 5155 to display an image of an operation site based on the image signal on which the image processing is performed by the image processing unit 5175. In this case, the control unit 5177 recognizes various objects in the operation site image by using various image recognition technologies. For example, the control unit 5177 detects the edge shape, color, and the like of each object included in the operation site image to recognize, for example, an operation instrument such as a forceps, a particular biological site, bleeding, and mist when the energy treatment instrument 5135 is used. When causing the display device 5155 to display the operation site image, the control unit 5177 uses a result of the recognition to display various kinds of operation support information on the operation site image in a superimposing manner. When the operation support information is displayed in a superimposing manner and presented to the operator 5181, a medical operation can be performed in a safer and more reliable manner.

The transmission cable 5179 connecting the camera head 5119 and the CCU 5153 is an electric signal cable compatible with electric signal communication, an optical fiber compatible with optical communication, or a composite cable thereof.

In the illustrated example, communication is performed in a wired manner by using the transmission cable 5179, but communication between the camera head 5119 and the CCU 5153 may be performed in a wireless manner. When communication therebetween is performed in a wireless manner, the transmission cable 5179 does not need to be laid in the medical operation room, which can eliminate a situation in which movement of medical staff in the medical operation room is interfered by the transmission cable 5179.

The above description is made on an example of the medical operation room system 5100 to which the technology according to the present disclosure is applicable. In this example, a medical system to which the medical operation room system 5100 is applied is the endoscope medical operation system 5113, but the configuration of the medical operation room system 5100 is not limited to the example. For example, the medical operation room system 5100 may be applied to an examination flexible endoscope system or a microscope medical operation system in place of the endoscope medical operation system 5113.

### 2. Exemplary Configuration of Support Arm Device

The following describes an exemplary configuration of a support arm device to which the technology according to the present disclosure is applicable. The support arm device described below is an exemplary support arm device configured to support the endoscope at the leading end of an arm unit, but the present embodiment is not limited to such an example. When the support arm device according to the embodiment of the present disclosure is applied to a medical field, the support arm device can function as a medical support arm device.

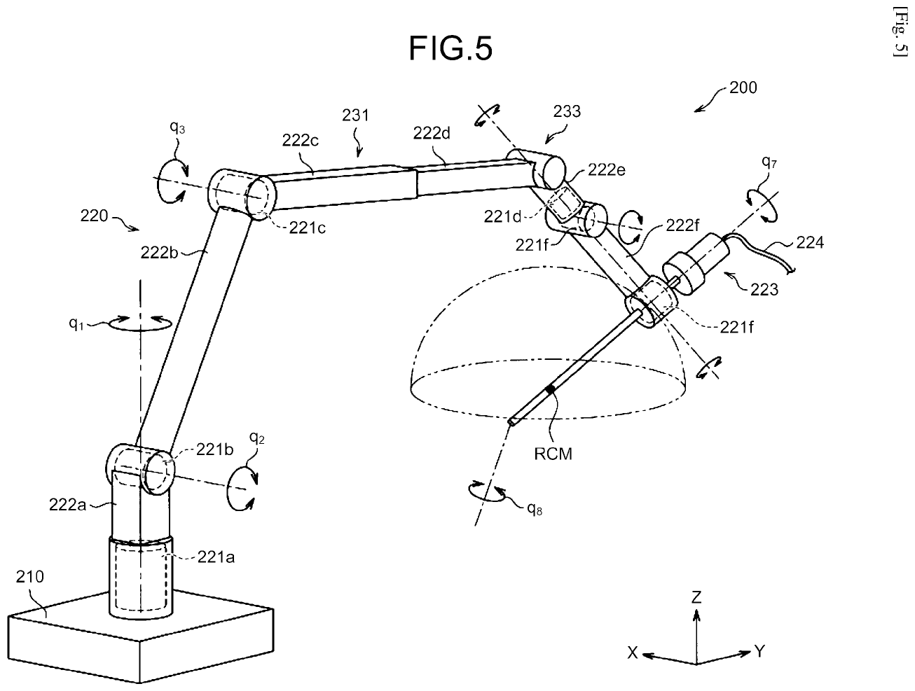

FIG. 5 is a schematic view illustrating the appearance of a support arm device 200 according to the present embodiment. As illustrated in FIG. 5, the support arm device 200 according to the present embodiment includes a base unit 210 and an arm unit 220. The base unit 210 is a base of the support arm device 200, and the arm unit 220 extends from the base unit 210. Although not illustrated in FIG. 5, a control unit configured to integrally control the support arm device 200 may be provided in the base unit 210, and drive of the arm unit 220 may be controlled by the control unit. The control unit is achieved by various signal processing circuits such as a CPU and a DSP.

The arm unit 220 includes a plurality of active joint parts 221a to 221f, a plurality of links 222a to 222f, and an endoscope device 223 as a leading end unit provided at the leading end of the arm unit 220.

The links 222a to 222f are members having substantially bar shapes. One end of the link 222a is coupled with the base unit 210 through the active joint part 221a, the other end of the link 222a is coupled with one end of the link 222b through the active joint part 221b, and the other end of the link 222b is coupled with one end of the link 222c through the active joint part 221c. The other end of the link 222c is coupled with the link 222d through a passive slide mechanism 231, and the other end of the link 222d is coupled with one end of the link 222e through a passive joint part 233. The other end of the link 222e is coupled with one end of the link 222f through the active joint parts 221d and 221e. The endoscope device 223 is coupled with the leading end of the arm unit 220, in other words, the other end of the link 222f through the active joint part 221f. In this manner, the ends of the links 222a to 222f are coupled with each other through the active joint parts 221a to 221f, the passive slide mechanism 231, and the passive joint part 233 with the base unit 210 as a pivot, thereby forming an arm shape extending from the base unit 210.

The position and posture of the endoscope device 223 are controlled as drive of an actuator provided to each of the active joint parts 221a to 221f of the arm unit 220 is controlled. In the present embodiment, the leading end of the endoscope device 223 enters the body cavity of the patient as a treatment site and captures an image of a partial region of the treatment site. However, the leading end unit provided at the leading end of the arm unit 220 is not limited to the endoscope device 223, but an exoscope or a microscope may be used in place of the endoscope. The leading end of the arm unit 220 may be connected with various medical instruments as leading end units. In this manner, the support arm device 200 according to the present embodiment is configured as a medical support arm device including a medical instrument.

The following describes the support arm device 200 based on definition of coordinate axes as illustrated in FIG. 5. In addition, an up-down direction, a front-back direction, and a right-left direction are defined in accordance with the coordinate axes. Specifically, a z-axial direction and the up-down direction are defined to be an up-down direction with respect to the base unit 210 installed on a floor surface. In addition, a y-axial direction and the front-back direction are defined to be a direction that is orthogonal to the z-axis and in which the arm unit 220 extends from the base unit 210 (in other words, a direction in which the endoscope device 223 is positioned relative to the base unit 210). In addition, an x-axial direction and the right-left direction are defined to be a direction orthogonal to the y-axis and the z-axis.

The active joint parts 221a to 221f each couple links in a manner rotatable relative to each other. The active joint parts 221a to 221f each include a rotation mechanism including an actuator and configured to be rotated about a predetermined rotational axis through drive of the actuator. Drive of the arm unit 220 such as stretching and contracting (folding) of the arm unit 220 can be controlled by controlling rotation at each of the active joint parts 221a to 221f. Drive of the active joint parts 221a to 221f may be controlled by, for example, well-known body cooperative control and ideal joint control. Since the active joint parts 221a to 221f each include the rotation mechanism as described above, drive control of the active joint parts 221a to 221f in the following description specifically means control of the rotation angles and/or generated torque (torque generated by the active joint parts 221a to 221f) of the active joint parts 221a to 221f.

The passive slide mechanism 231 is an aspect of a passive form change mechanism and couples the links 222c and 222d so that the links are movable relative to each other in a predetermined direction. For example, the passive slide mechanism 231 may couple the links 222c and 222d so that the links are movable straight relative to each other. However, the movement of the links 222c and 222d is not limited to straight motion but may be motion in a direction along an arc. An operation for the movement is performed on the passive slide mechanism 231, for example, by the user, and the distance between the active joint part 221c on one end side of the link 222c and the passive joint part 233 is variable. Accordingly, the entire form of the arm unit 220 can be changed.

The passive joint part 233 is an aspect of a passive form change mechanism and couples the links 222d and 222e in a manner rotatable relative to each other. An operation for the rotation is performed on the passive joint part 233, for example, by the user, and the angle between the links 222d and 222e is variable. Accordingly, the entire form of the arm unit 220 can be changed.

In the present specification, “the posture of the arm unit” means the state of the arm unit in which at least some parts of an arm can be changed by drive control or the like. As a specific example, “the posture of the arm unit” is the state of the arm unit that can be changed through drive control of the actuators provided to the active joint parts 221a to 221f by the control unit while the distance between active joint parts adjacent to each other with one or a plurality of links interposed therebetween is fixed. In the present disclosure, “the posture of the arm unit” is not limited to the state of the arm unit that can be changed through drive control of the actuators. For example, “the posture of the arm unit” may be the state of the arm unit that is changed through cooperative operation of passive joint parts. In the present disclosure, the arm unit does not necessarily need to include a joint part. In this case, “the posture of the arm unit” is a position relative to an object or a relative angle relative to the object. “The form of the arm unit” means the state of the arm unit that can be changed as the relation in position and posture between components of the arm is changed. As a specific example, “the form of the arm unit” is the state of the arm unit that can be changed as the distance between active joint parts adjacent to each other with a link interposed therebetween and the angle between links each connecting active joint parts adjacent to each other are changed along with an operation of a passive form change mechanism. In the present disclosure, “the form of the arm unit” is not limited to the state of the arm unit that can be changed as the distance between active joint parts adjacent to each other with a link interposed therebetween and the angle between links each connecting active joint parts adjacent to each other are changed. For example, “the form of the arm unit” may be the state of the arm unit that can be changed as the positional relation and angle between the passive joint parts are changed through cooperative operation of the passive joint parts. When the arm unit includes no joint part, “the posture of the arm unit” may be the state of the arm unit that can be changed as a position relative to an object and a relative angle relative to the object are changed.

The support arm device 200 according to the present embodiment includes the six active joint parts 221a to 221f and has six degrees of freedom for drive of the arm unit 220. Thus, drive control of the support arm device 200 is achieved through drive control of the six active joint parts 221a to 221f by the control unit, but drive control of the passive slide mechanism 231 and the passive joint part 233 is not performed by the control unit.

Specifically, as illustrated in FIG. 5, the active joint parts 221a, 221d, and 221f are provided so that the rotational axis directions thereof are aligned with the longitudinal axial directions of the links 222a and 222e connected therewith and the image capturing direction of the endoscope device 223 connected therewith. The active joint parts 221b, 221c, and 221e are provided so that the rotational axis directions thereof are aligned with the x-axial direction as a direction in which the coupling angles of the links 222a to 222c, 222e, and 222f and the endoscope device 223 connected therewith are changed in a y-z plane (plane defined by the y-axis and the z-axis). In this manner, in the present embodiment, the active joint parts 221a, 221d, and 221f have functions to perform what is called yawing, and the active joint parts 221b, 221c, and 221e have functions to perform what is called pitching.

With such a configuration of the arm unit 220, six degrees of freedom are obtained for drive of the arm unit 220 in the support arm device 200 according to the present embodiment, and thus the endoscope device 223 can be freely moved in the movable range of the arm unit 220. FIG. 3 illustrates a hemisphere as an exemplary movable range of the endoscope device 223. When the central point RCM (remote center of motion) of the hemisphere is an image capturing center of a treatment site, an image of which is captured by the endoscope device 223, image capturing of the treatment site can be performed at various angles by moving the endoscope device 223 on the spherical surface of the hemisphere while the image capturing center of the endoscope device 223 is fixed to the central point of the hemisphere.

The description so far is made on the exemplary configuration of the support arm device to which the technology according to the present disclosure is applicable.

In the above description, the arm unit 220 of the support arm device 200 includes a plurality of joint parts and has six degrees of freedom, but the present disclosure is not limited thereto. Specifically, it suffices that the arm unit 220 is provided with the endoscope device 223 or an exoscope at the leading end. For example, the arm unit 220 may have only one degree of freedom for driving the arm unit 220 to move in a direction in which the endoscope device 223 enters and retracts from the body cavity of the patient.

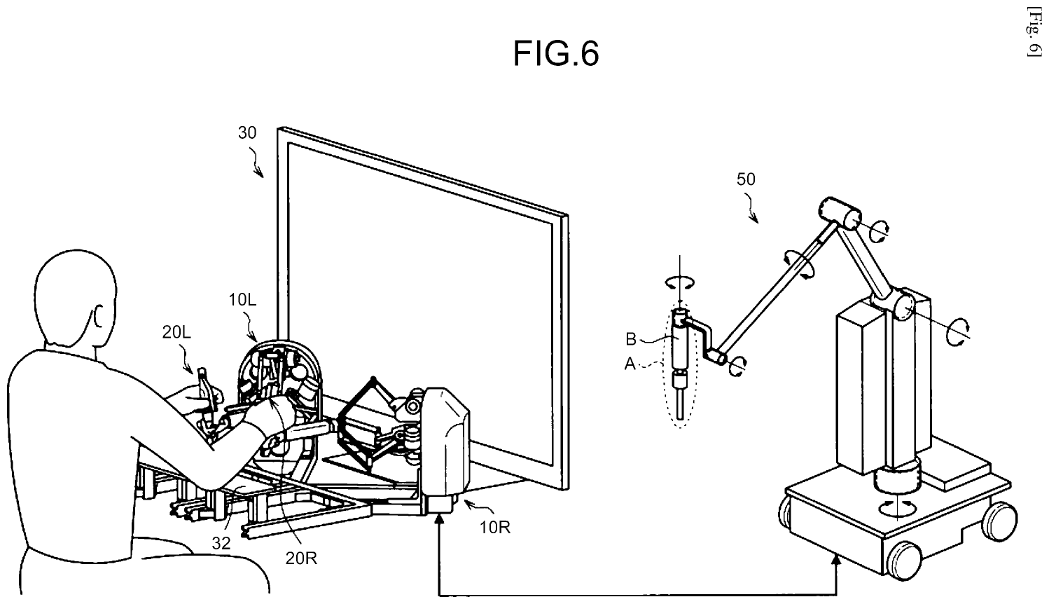

The present disclosure may be a master-slave apparatus as illustrated in FIG. 6.

A master device 10 is an information processing device (first information processing device) having functions to control drive of a slave device 50 and present a vibration signal (first signal) measured by a sensor of the slave device 50 to the user. The master device 10 is, for example, a device including one or a plurality of joints including a passive joint, and a link connected with each joint (device including a link mechanism including a passive joint). The passive joint is a joint that is not driven by a motor, an actuator, or the like.

As illustrated in FIG. 6, the master device 10 includes operation devices 20 (20R and 20L) grasped and operated by the user. The operation devices 20 correspond to a tactile presentation device according to the embodiment of the present disclosure. The master device 10 is connected with a monitor 30 on which an operative field is displayed, and is provided with a support table 32 on which the user places both arms or both elbows. The master device 10 includes a right-hand master device 10R and a left-hand master device 10L. The right-hand master device 10R includes the right-hand operation device 20R, and the left-hand master device 10L includes the left-hand operation device 20L.

The user places both arms or both elbows on the support table 32 and grasps the operation devices 20R and 20L with the right hand and the left hand, respectively. In this state, the user operates the operation devices 20R and 20L while watching the monitor 30 on which the operative field is displayed. The user may remotely operate the position or orientation of an operation instrument attached to the slave device 50 by displacing the position or orientation of each of the operation devices 20R and 20L, or may perform a grasping operation with each operation instrument.

The slave device 50 is an information processing device (second information processing device) configured to present, to the master device 10, force and vibration generated when an affected part (hereinafter also referred to as an object) of a patient in a medical operation contacts a site of the slave device 50 that contacts the object. For example, the slave device 50 is a device including one or a plurality of active joints and a link connected with each active joint for moving in accordance with movement of the master device 10 (device including a link mechanism including an active joint). The active joint is a joint that is driven by a motor, an actuator, or the like.

In the slave device 50, various sensors as examples of external device (such as an location sensor, a limit sensor, an encoder such as a rotary or linear encoder, a microphone, and an acceleration sensor) are provided at a leading end part (part A illustrated in FIG. 6) of an arm illustrated in FIG. 6. In addition, a force sensor (part B illustrated in FIG. 6) is provided at the leading end part of the arm of the slave device 50. The force sensor measures force applied to the leading end part of the arm when the leading end part of the arm contacts the patient. Places at which the above-described various sensors are provided are not particularly limited, but the various sensors may be provided at optional places of the leading end part of the arm.

For example, the slave device 50 includes, at a position corresponding to each active joint, a motion sensor for measuring motion of the active joint. The above-described motion sensor is, for example, an encoder. In addition, for example, the slave device 50 includes, at a position corresponding to each active joint, a drive mechanism for driving the active joint. The above-described drive mechanism is, for example, a motor or a driver.

The embodiment of the present disclosure may be applied to a virtual reality environment. For example, when the master device 10 is operated, a video of a virtual environment on the slave device 50 side may be displayed on the monitor 30 so that the user operates the master device 10 based on the video.

### 3. Medical Operation Support System

(3-1. Configuration of Medical Operation Support System)

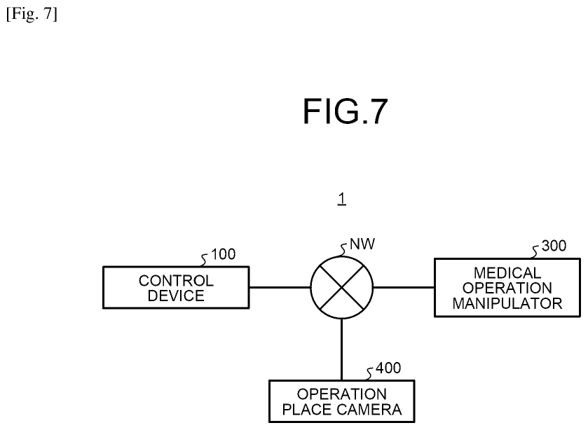

The following describes the configuration of a medical operation support system according to the embodiment of the present disclosure with reference to FIG. 7. FIG. 7 is a diagram illustrating an exemplary configuration of the medical operation support system according to the embodiment of the present disclosure.

As illustrated in FIG. 7, this medical operation support system 1 (computer-supported medical operation system) includes a control device 100, a medical operation manipulator 300, and an operation place camera 400. The control device 100 and the medical operation manipulator 300 are connected with each other through a network NW to perform communication therebetween. The operation place camera 400 is connected with at least the control device 100 to perform communication therebetween. The medical operation manipulator 300 is autonomously or semi-autonomously driven to provide various kinds of medical treatment on a patient. In the present embodiment, the control device 100 performs switching between autonomous drive (may include semi-autonomous drive) of the medical operation manipulator 300 and a manual operation. The medical operation manipulator 300, which autonomously or semi-autonomously executes various kinds of medical treatment on the patient, is also called a medical operation robot.

(3-2. Medical Operation Manipulator)

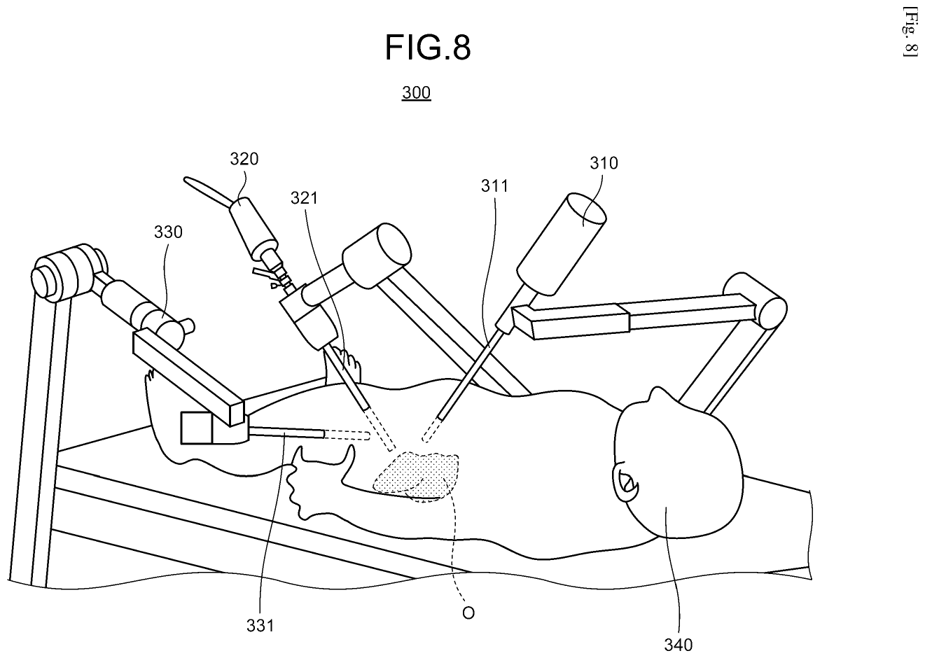

The following describes an exemplary configuration of the medical operation manipulator with reference to FIG. 8. FIG. 8 is a diagram illustrating an exemplary configuration of the medical operation manipulator.

As illustrated in FIG. 8, the medical operation manipulator 300 includes a first support arm device 310, a second support arm device 320, and a third support arm device 330. The first support arm device 310 is provided with a first medical instrument 311. The second support arm device 320 is provided with a second medical instrument 321. The third support arm device 330 is provided with a third medical instrument 331. For example, the first support arm device 310 to the third support arm device 330 have configurations same as that of the support arm device 200 illustrated in FIG. 5, but the present disclosure is not limited thereto. For example, the first support arm device 310 to the third support arm device 330 only need to have configurations with which the first support arm device 310 to the third support arm device 330 can support the first medical instrument 311 to the third medical instrument 331, respectively. The medical operation manipulator 300 may also include another support arm device.

The medical operation manipulator 300 provides various kinds of medical treatment on a patient 340 cooperatively with a doctor (or a team including an operating surgeon and a support staff) by using the first medical instrument 311 and the second medical instrument 321. The third medical instrument 331 is, for example, an endoscope device and captures an image of the situation of the body cavity of the patient 340. For example, the third medical instrument 331 captures an image of the situation of an organ O of the patient 340.

The medical operation manipulator 300 acquires instrument information for determining the kinds of the first medical instrument 311 to the third medical instrument 331. For example, each medical instrument may be electrically provided with identification information in accordance with the kind thereof, and the medical operation manipulator 300 may determine the kind by reading the identification information when the medical instrument is supported by the first support arm device 310 to the third support arm device 330.

The medical operation manipulator 300 acquires external force information related to external force applied from the outside. For example, the medical operation manipulator 300 acquires external force information related to external force applied from the outside to the first support arm device 310 to the third support arm device 330. In this case, for example, the first support arm device 310 to the third support arm device 330 may be each provided with an acceleration sensor, and the magnitude of acceleration acquired by the acceleration sensor may be set as the magnitude of the external force.

(3-3. Control Device)

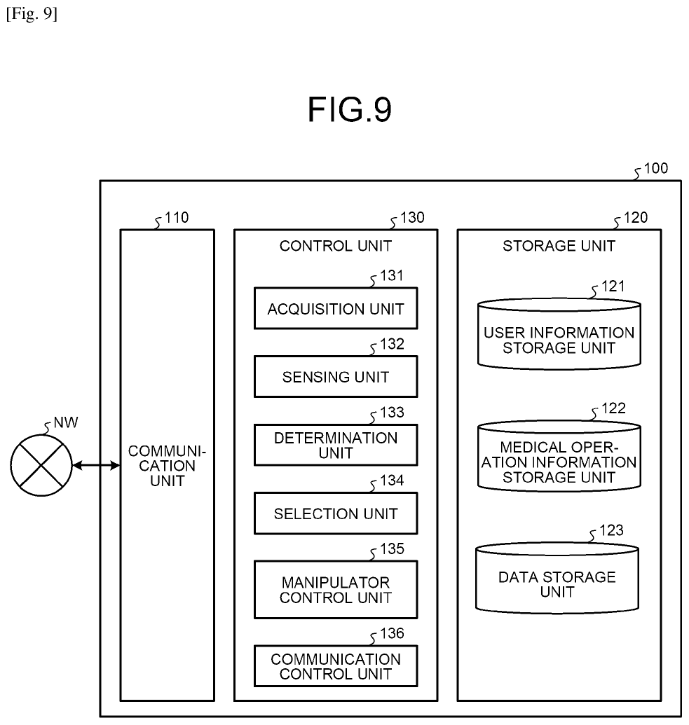

The following describes the configuration of the control device 100 with reference to FIG. 9. FIG. 9 is a block diagram illustrating an exemplary configuration of the control device 100.

As illustrated in FIG. 9, the control device 100 includes a communication unit 110, a storage unit 120, and a control unit 130.

The communication unit 110 is achieved by, for example, a network interface card (NIC) or a communication circuit. The communication unit 110 is connected with the network NW (such as the Internet) in a wired or wireless manner. Through the network NW, the communication unit 110 transmits and receives information to and from another device or the like in accordance with control of a communication control unit 136. For example, the communication unit 110 transmits and receives information to and from the medical operation manipulator 300. For example, the communication unit 110 transmits and receives information to and from the operation place camera 400.

The storage unit 120 is achieved by a storage device such as a semiconductor memory element including a random access memory (RAM) and a flash memory, a hard disk, or an optical disk. The storage unit 120 includes a user information storage unit 121, a medical operation information storage unit 122, and a data storage unit 123.

The user information storage unit 121 stores various kinds of information to be used to identify a user. The user information storage unit 121 stores various kinds of information for identifying a doctor or the like operating the medical operation manipulator 300. The user information storage unit 121 stores image data of the face of each user, voice data thereof, and the like for identifying the user. The user information storage unit 121 may also store biological information for identifying a user and other identification information.

The medical operation information storage unit 122 stores information related to various medical operations (medical treatment) performed by the medical operation manipulator 300. The medical operation information storage unit 122 stores information related to a process (schedule or procedure) between the start and end of each of the various medical operations. The medical operation information storage unit 122 stores, for example, information related to time taken by the medical operation manipulator 300 for a treatment.

The data storage unit 123 stores various kinds of data. The data storage unit 123 may store, for example, results of various kinds of determinations by a determination unit 133.

The control unit 130 is achieved by, for example, a central processing unit (CPU) or a micro processing unit (MPU) executing a computer program (for example, an information processing program according to the present disclosure) stored in the control device 100 by using a random access memory (RAM) or the like as a work area. The control unit 130 is a controller and may be achieved by an integrated circuit such as an application specific integrated circuit (ASIC) or a field programmable gate array (FPGA). The control unit 130 includes an acquisition unit 131, a sensing unit 132, the determination unit 133, a selection unit 134, a manipulator control unit 135, and the communication control unit 136.

The acquisition unit 131 acquires various kinds of information. The acquisition unit 131 acquires various kinds of information from, for example, the medical operation manipulator 300. The acquisition unit 131 acquires, from the medical operation manipulator 300, for example, operation information related to an operation on the medical operation manipulator 300 by the operator. The acquisition unit 131 acquires, from the medical operation manipulator 300, for example, instrument information related to medical instruments mounted on the first support arm device 310 to the third support arm device 330. The acquisition unit 131 acquires, from the medical operation manipulator 300, for example, external force information related to external force applied to the medical operation manipulator 300. The acquisition unit 131 acquires, from the medical operation manipulator 300, for example, external force information related to external force applied to the first support arm device 310 to the third support arm device 330. The acquisition unit 131 acquires, from the medical operation manipulator 300, for example, image data obtained through image capturing of the situation of the body cavity of a patient.

The acquisition unit 131 acquires various kinds of information from the operation place camera 400. The acquisition unit 131 acquires, for example, image data obtained through image capturing of the medical operation room from the operation place camera 400. The acquisition unit 131 acquires, for example, image data obtained through image capturing of the medical operation manipulator 300. The acquisition unit 131 acquires, for example, image data obtained through image capturing of medical instruments provided to the first support arm device 310 to the third support arm device 330. The acquisition unit 131 acquires, for example, image data obtained through image capturing of the operator of the medical operation manipulator 300.

The sensing unit 132 senses various kinds of information. The sensing unit 132 senses various kinds of information based on, for example, information acquired by the acquisition unit 131. The sensing unit 132 senses, based on, for example, operation information related to an operation on the medical operation manipulator 300 acquired by the acquisition unit 131, that the medical operation manipulator 300 receives an operation to perform switching between an autonomous drive mode in which the medical operation manipulator 300 is autonomously driven and a manual operation mode in which the medical operation manipulator 300 is manually operated.

The determination unit 133 determines various kinds of information. The determination unit 133 determines, for example, whether the state of the medical operation manipulator 300 is in the autonomous drive mode or the manual operation mode. The determination unit 133 determines various kinds of information, for example, when the sensing unit 132 senses an operation that performs switching between the autonomous drive mode and the manual operation mode is performed on the medical operation manipulator 300. In this case, the determination unit 133 determines various kinds of information based on, for example, information acquired by the acquisition unit 131. The determination unit 133 determines the status based on, for example, image data acquired by the acquisition unit 131. The determination unit 133 determines the state of the medical operation manipulator 300 based on, for example, the image data. The determination unit 133 determines the state of the operator of the medical operation manipulator 300 based on, for example, the image data. The determination unit 133 determines the status of a medical operation based on, for example, the image data. The determination unit 133 determines the status based on, for example, operation information acquired by the acquisition unit 131. The determination unit 133 performs the determination based on, for example, a record of biological information such as the degree of fatigue of the operator of the medical operation manipulator 300.

The selection unit 134 selects various kinds of information. The selection unit 134 selects various kinds of information based on, for example, a result of the determination by the determination unit 133. The selection unit 134 selects a switching sequence for switching the autonomous drive mode and the manual operation mode of the medical operation manipulator 300 based on, for example, the status determined by the determination unit 133.

The manipulator control unit 135 controls various states of the medical operation manipulator 300. The manipulator control unit 135 controls the medical operation manipulator 300 based on, for example, a result of the selection by the selection unit 134. The manipulator control unit 135 switches the autonomous drive mode and the manual operation mode of the medical operation manipulator 300 in accordance with, for example, the switching sequence selected by the selection unit 134.

The communication control unit 136 controls transmission and reception of information through the communication unit 110. The communication control unit 136 controls the communication unit 110 to perform communication with another information processing device. The communication control unit 136 controls the communication unit 110 to perform communication with, for example, the medical operation manipulator 300. The communication control unit 136 controls the communication unit 110 to perform communication with, for example, the operation place camera 400.

### 4. Processing at Control Device

(4-1. First Processing)

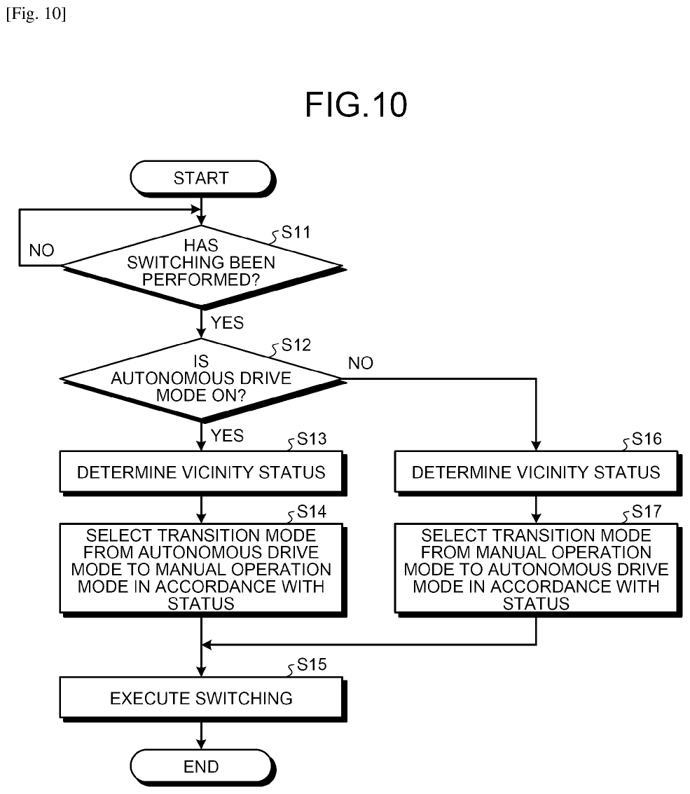

The following describes first processing at the control unit 130 of the control device 100 according to the embodiment of the present disclosure with reference to FIG. 10. FIG. 10 is a flowchart illustrating the process of the first processing at the control unit 130 of the control device 100 according to the embodiment of the present disclosure.

First, the control unit 130 determines whether an operation-mode switching operation has been performed on the medical operation manipulator 300 (step S11). Specifically, the determination is performed as the sensing unit 132 senses the operation-mode switching operation based on operation information acquired by the acquisition unit 131. When it is determined that the switching operation has not been performed (No at step S11), the control unit 130 repeats the processing at step S11. When it is determined that the switching operation has been performed (Yes at step S11), the process proceeds to step S12. The system switches modes when it determines the surgical scene is safe enough for the transition from one control to the next. For example, the distance between the surgical instrument and the organ is confirmed if the distance is far enough. The position of the operator may also be considered. When switching from a less autonomous mode to a more autonomous mode, a transition process is set for a period of time (e.g., a few seconds) prior to executing the change. This period affords the attendant (e.g., surgeon) a sufficient time to consider whether the attendant wishes to override the mode change. If so, the attendant can apply a force to the medical operation manipulator, or simply move his or her hand near the surgical site so system detects that the surgical scene has changed and thus halts the transition. On the other hand, when switching back from more autonomous mode to the less autonomous mode, the system confirms the operator is ready for taking control of the medical operation manipulator before it changes the operation mode. To do so, the systems set a constraint on the system where the operator can move the medical operation manipulator, for example, only in a restricted direction and confirms if the operator is ready for the switch, then the constraint is removed (by degree). The system optionally has a different type/direction of the constraint depending on the medical tool being used or the state of the surgical operation.

When positive determination is made at step S11, the control unit 130 determines whether the state of the medical operation manipulator 300 is in the autonomous drive mode (step S12). Specifically, the determination unit 133 determines whether the autonomous drive mode is on. When it is determined that the medical operation manipulator 300 is in the autonomous drive mode (Yes at step S12), the process proceeds to step S13. When it is determined that the medical operation manipulator 300 is in the manual operation mode (No at step S12), the process proceeds to step S16.

When positive determination is made at step S12, the control unit 130 determines the status of the vicinity of the medical operation manipulator 300 (step S13). Specifically, the determination unit 133 determines the status of the vicinity of the medical operation manipulator 300 based on environment information such as image data of the medical operation room acquired by the acquisition unit 131. Specifically, the status of the medical operation room includes various kinds of information related to the medical operation room, such as the state of the medical operation manipulator 300, people (including a doctor and a patient) in the medical operation room, and the state (including the position) of people in the medical operation room. Then, the process proceeds to step S14.

The control unit 130 selects a transition mode in which transition is performed from the autonomous drive mode to the manual operation mode in accordance with the status of the vicinity of the medical operation manipulator 300 (step S14). Specifically, the selection unit 134 selects the transition mode based on the result of the determination by the determination unit 133. Thus, the selection unit 134 can select a different transition mode in accordance with the status of the vicinity. In other words, the autonomous drive mode and the manual operation mode are switched through a switching sequence that differs in accordance with the status of the vicinity. Then, the process proceeds to step S15.

The control unit 130 switches the autonomous drive mode and the manual operation mode of the medical operation manipulator 300 (step S15). Specifically, the manipulator control unit 135 switches the autonomous drive mode and the manual operation mode based on a result of the selection by the selection unit 134. Then, the processing illustrated in FIG. 10 ends.

When negative determination is made at step S12, the control unit 130 determines the status of the vicinity of the medical operation manipulator 300 (step S16). This processing is the same as that at step S13, and thus description thereof is omitted. Then, the process proceeds to step S17.

The control unit 130 selects a transition mode in which transition is performed from the manual operation mode to the autonomous drive mode in accordance with the status of the vicinity of the medical operation manipulator 300 (step S17). Specifically, the selection unit 134 selects the transition mode based on the result of the determination by the determination unit 133. Then, the process proceeds to step S15.

As described above, in the present embodiment, a transition mode can be selected in which the autonomous drive mode and the manual operation mode are switched in accordance with the status of the vicinity of the medical operation manipulator 300. Accordingly, the autonomous drive mode and the manual operation mode of the medical operation manipulator 300 can be optimally switched.

(4-2. Second Processing)

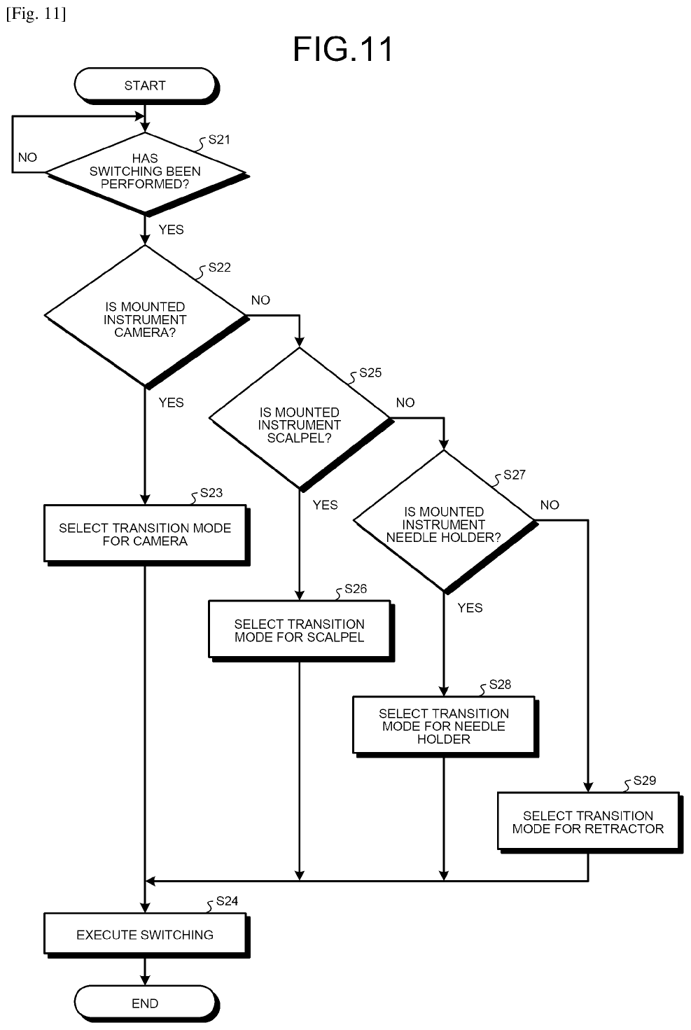

The following describes second processing at the control unit 130 of the control device 100 according to the embodiment of the present disclosure with reference to FIG. 11. FIG. 11 is a flowchart illustrating the process of the second processing at the control unit 130 of the control device 100 according to the embodiment of the present disclosure.

Depending on the kind of a medical instrument, changing a switching sequence is assumed to be preferable, and thus the second processing executes processing of selecting a transition mode in accordance with a medical instrument mounted on the medical operation manipulator 300. In the second processing, the autonomous drive mode and the manual operation mode are switched through a switching sequence that differs in accordance with the kind of medical instrument. The following description is made for a case in which each of a camera (for example, an endoscope), a scalpel, a needle holder, and a retractor is mounted on the medical operation manipulator 300, but the present disclosure is not limited to the case. In addition, the order of instrument check is presented in description of FIG. 11, but the present disclosure is not limited thereto.

Step S21 is the same as step S11 illustrated in FIG. 10, and thus description thereof is omitted.

When positive determination is made at step S21, the control unit 130 determines whether the medical instrument mounted on the medical operation manipulator 300 is a camera (step S22). Specifically, the determination unit 133 determines whether the medical instrument is a camera based on image data of the medical operation manipulator 300 acquired by the acquisition unit 131. When the acquisition unit 131 has acquired instrument information from the medical operation manipulator 300, the determination unit 133 determines whether the medical instrument is a camera based on the instrument information. When it is determined that the medical instrument is a camera (Yes at step S22), the process proceeds to step S23. When it is determined that the medical instrument is not a camera (No at step S22), the process proceeds to step S25.

When positive determination is made at step S22, the control unit 130 selects a transition mode for the camera (step S23). Specifically, the selection unit 134 selects the transition mode for the camera. Then, the process proceeds to step S24.

The control unit 130 switches the autonomous drive mode and the manual operation mode of the medical operation manipulator 300 (step S24). Specifically, the manipulator control unit 135 switches the autonomous drive mode and the manual operation mode based on a result of the selection by the selection unit 134. Then, the processing illustrated in FIG. 11 ends.

When negative determination is made at step S22, the control unit 130 determines whether the medical instrument mounted on the medical operation manipulator 300 is a scalpel (step S25). The method of the determination is the same as that for step S22, and thus description thereof is omitted. When it is determined that the medical instrument is a scalpel (Yes at step S25), the process proceeds to step S26. When it is determined that the medical instrument is not a scalpel (No at step S25), the process proceeds to step S27.

When positive determination is made at step S25, the control unit 130 selects a transition mode for the scalpel (step S26). Specifically, the selection unit 134 selects the transition mode for the scalpel. Then, the process proceeds to step S24.

When negative determination is made at step S25, the control unit 130 determines whether the medical instrument mounted on the medical operation manipulator 300 is a needle holder (step S27). The method of the determination is the same as that for step S22, and thus description thereof is omitted. When it is determined that the medical instrument is a needle holder (Yes at step S27), the process proceeds to step S28. When it is determined that the medical instrument is not a needle holder (No at step S27), the process proceeds to step S29.

When positive determination is made at step S27, the control unit 130 selects a transition mode for the needle holder (step S28). Specifically, the selection unit 134 selects the transition mode for the needle holder. Then, the process proceeds to step S24.

When negative determination is made at step S27, the control unit 130 selects a transition mode for the retractor (step S29). Specifically, the selection unit 134 selects the transition mode for the retractor. Then, the process proceeds to step S24.

(4-2-1. Processing in a Case of Camera)

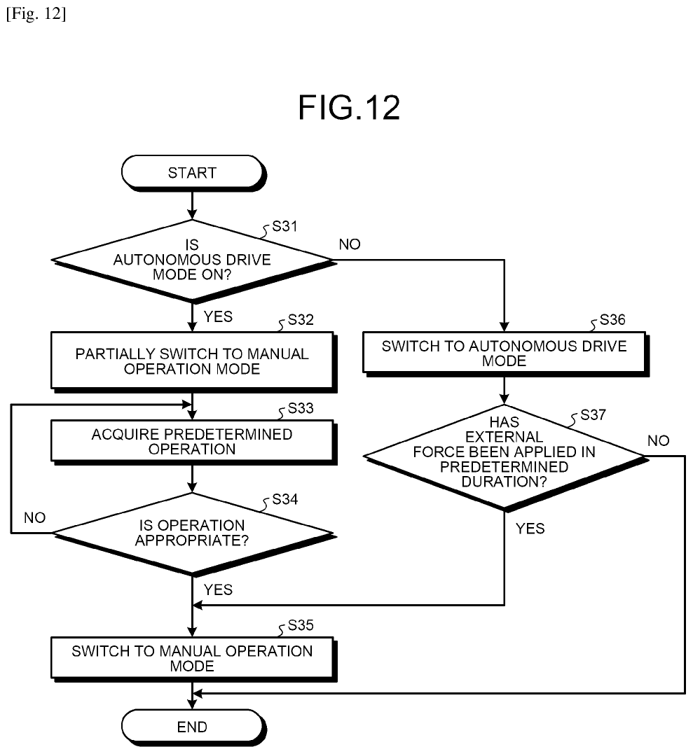

The following describes switching processing performed when the medical instrument mounted on the medical operation manipulator 300 is a camera such as an endoscope or a microscope with reference to FIG. 12. FIG. 12 is a flowchart illustrating an exemplary process of switching processing performed when the medical instrument mounted on the medical operation manipulator 300 is a camera.

The control unit 130 determines whether the state of the medical operation manipulator 300 is in the autonomous drive mode (step S31). Specifically, the determination unit 133 determines whether the autonomous drive mode is on. When it is determined that the medical operation manipulator 300 is in the autonomous drive mode (Yes at step S31), the process proceeds to step S32. When it is determined that the medical operation manipulator 300 is in the manual operation mode (No at step S31), the process proceeds to step S36.

When positive determination is made at step S31, the control unit 130 switches part of the medical operation manipulator 300 to the manual operation mode (step S32). Specifically, the manipulator control unit 135 performs switching to a state in which a part where the camera is mounted at the leading end is fixed and a basal side part (for example, a joint part) of the camera can be driven by the operator. Accordingly, the camera can be prevented from moving when switching is performed to the manual operation mode, and thus the visual field of a doctor performing manipulation can be maintained. Then, the process proceeds to step S33.

The control unit 130 acquires a predetermined operation on the medical operation manipulator 300 (step S33). Specifically, the acquisition unit 131 acquires operation information related to the predetermined operation on the medical operation manipulator 300. Then, the process proceeds to step S34.

The control unit 130 determines whether the predetermined operation on the medical operation manipulator 300 is appropriate (step S34). Specifically, the determination unit 133 determines, based on the operation information acquired by the acquisition unit 131, whether the doctor has been manually operating the basal side part of the camera that can be driven. When it is determined that the operation is appropriate (Yes at step S34), the process proceeds to step S35. When it is determined that the operation is inappropriate (No at step S34), the process proceeds to step S33.

When positive determination is made at step S34, the control unit 130 switches the medical operation manipulator 300 to the manual operation mode (step S35). Specifically, the manipulator control unit 135 switches the medical operation manipulator 300 to the manual operation mode. As described above, when switching is performed from the autonomous drive mode to the manual operation mode, improved safety can be achieved by performing the switching in stages. Then, the processing in FIG. 12 ends.

When negative determination is made at step S31, the control unit 130 switches the medical operation manipulator 300 to the autonomous drive mode (step S36). Specifically, the manipulator control unit 135 switches the medical operation manipulator 300 from the manual operation mode to the autonomous drive mode. Then, the process proceeds to step S37.

The control unit 130 determines whether external force has been applied to the medical operation manipulator 300 in a predetermined duration (step S37). Specifically, the determination unit 133 determines whether external force has been applied in the predetermined duration based on external force information acquired by the acquisition unit 131. The predetermined duration is not particularly limited but may be, for example, several seconds to several ten seconds. When it is determined that no external force has been applied in the predetermined duration (No at step S37), the processing in FIG. 12 ends. When it is determined that external force has been applied in the predetermined duration (Yes at step S37), the process proceeds to step S35. Accordingly, the medical operation manipulator 300 is switched to the manual operation mode, and thus safety can be maintained.

(4-2-2. Processing in a Case of Scalpel)

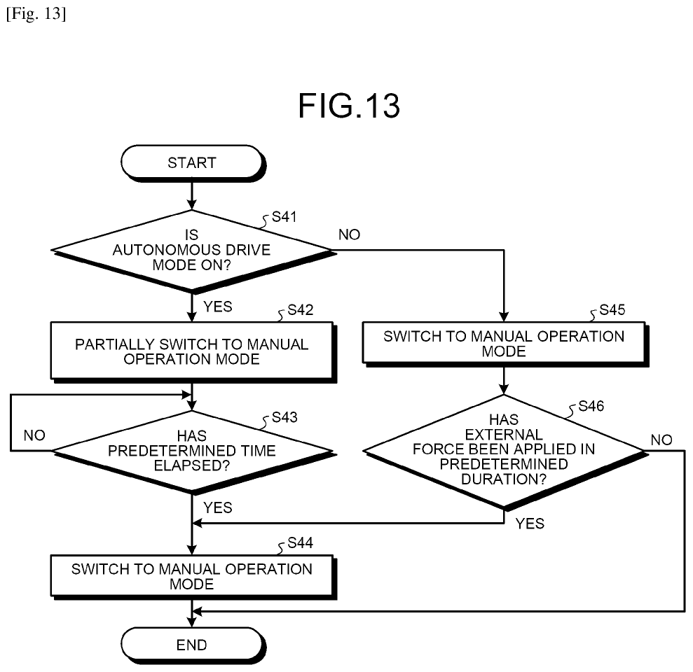

The following describes switching processing performed when the medical instrument mounted on the medical operation manipulator 300 is a scalpel with reference to FIG. 13. FIG. 13 is a flowchart illustrating an exemplary process of switching processing performed when the medical instrument mounted on the medical operation manipulator 300 is a scalpel.

Step S41 is the same as step S31 illustrated in FIG. 12, and thus description thereof is omitted.

When positive determination is made at step S41, the control unit 130 switches part of the medical operation manipulator 300 to the manual operation mode (step S42). Specifically, the manipulator control unit 135 performs switching to a state in which drive of the leading end part holding the scalpel is restricted to a direction in which the scalpel sticks into an organ (direction in which an operation instrument potentially damages a biological tissue) but the leading end part can be operated in other directions. The drive restriction may be performed only in a constant time. Accordingly, for example, it is possible to prevent damage to the body cavity with the scalpel through a false operation when switching is performed to the manual operation mode. As a result, improved safety can be achieved. Then, the process proceeds to step S43.

The control unit 130 determines whether a predetermined time has elapsed (step S43). Specifically, the determination unit 133 determines whether the predetermined time has elapsed. The predetermined time is a time in which, for example, switching to the manual operation mode can be sufficiently recognized by a doctor or the like. When it is determined that the predetermined time has not elapsed (No at step S43), the processing at step S43 is repeated. When the predetermined time has elapsed (Yes at step S43), the process proceeds to step S44.

When positive determination is made at step S43, the control unit 130 switches the medical operation manipulator 300 to the manual operation mode (step S44). Specifically, the manipulator control unit 135 switches the medical operation manipulator 300 to the manual operation mode. Then, the processing illustrated in FIG. 13 ends.

Steps S45 and S46 are same as steps S36 and S37 illustrated in FIG. 12, respectively, and thus description thereof is omitted.

(4-2-3. Processing in a Case of Needle Holder)

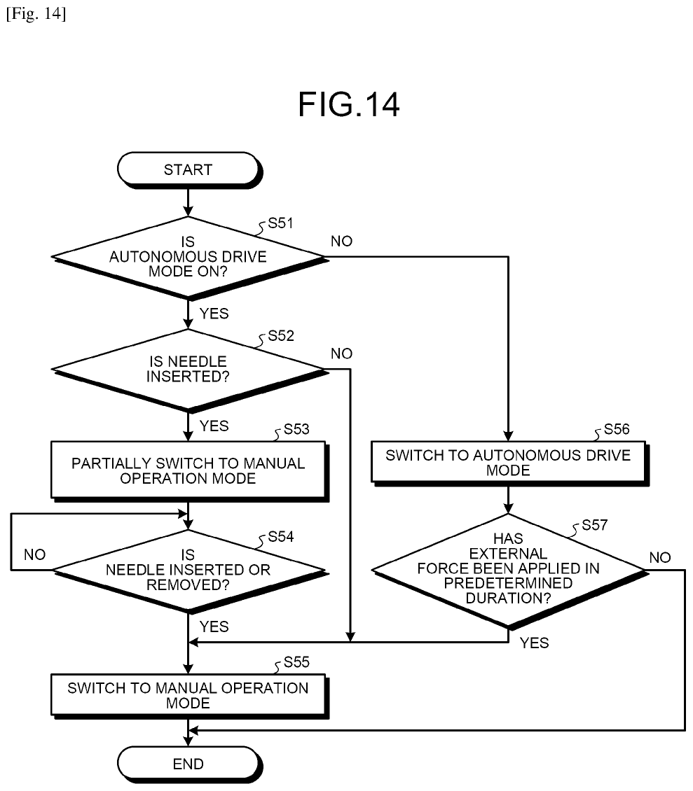

The following describes switching processing performed when the medical instrument mounted on the medical operation manipulator 300 is a needle holder with reference to FIG. 14. FIG. 14 is a flowchart illustrating an exemplary process of switching processing performed when the medical instrument mounted on the medical operation manipulator 300 is a needle holder.

Step S51 is the same as step S31 illustrated in FIG. 12, and thus description thereof is omitted.

When positive determination is made at step S51, the control unit 130 determines whether a needle grasped by the needle holder is inserted into an affected part (for example, an organ) (step S52). Specifically, the determination unit 133 determines whether the needle is inserted into the affected part based on image data of the operation site acquired by the acquisition unit 131. When it is determined that the needle is inserted into the affected part (Yes at step S52), the process proceeds to step S53. When it is determined that the needle is not inserted into the affected part (No at step S52), the process proceeds to step S55.

When positive determination is made at step S52, the control unit 130 switches part of the medical operation manipulator 300 to the manual operation mode (step S53). Specifically, the manipulator control unit 135 performs switching to a state in which a manual operation is possible only in the insertion-removal direction of the needle and a needle crossing direction when the operation site is sutured but the manual operation is restricted in the other directions. In other words, the operator cannot perform a manual operation in any direction other than the insertion-removal direction of the needle. Accordingly, for example, it is possible to prevent damage to an organ through a false operation when switching is performed to the manual operation mode. As a result, improved manipulation safety can be achieved. Then, the process proceeds to step S54.

The control unit 130 determines whether the needle is inserted into or removed from a treatment site (step S54). Specifically, the determination unit 133 determines whether the needle is inserted into or removed from the treatment site based on image data of the operation site acquired by the acquisition unit 131. When it is determined that the needle is inserted into or removed from the treatment site (Yes at step S54), the process proceeds to step S55. When it is determined that the needle is not inserted into or removed from treatment site (No at step S54), the processing at step S54 is repeated.

When positive determination is made at step S54, the control unit 130 switches the medical operation manipulator 300 to the manual operation mode (step S55). Specifically, the manipulator control unit 135 switches the medical operation manipulator 300 to the manual operation mode. Then, the processing illustrated in FIG. 14 ends.

Steps S56 and S57 are same as steps S36 and S37 illustrated in FIG. 12, respectively, and thus description thereof is omitted.

(4-2-4. Processing in a Case of Retractor)

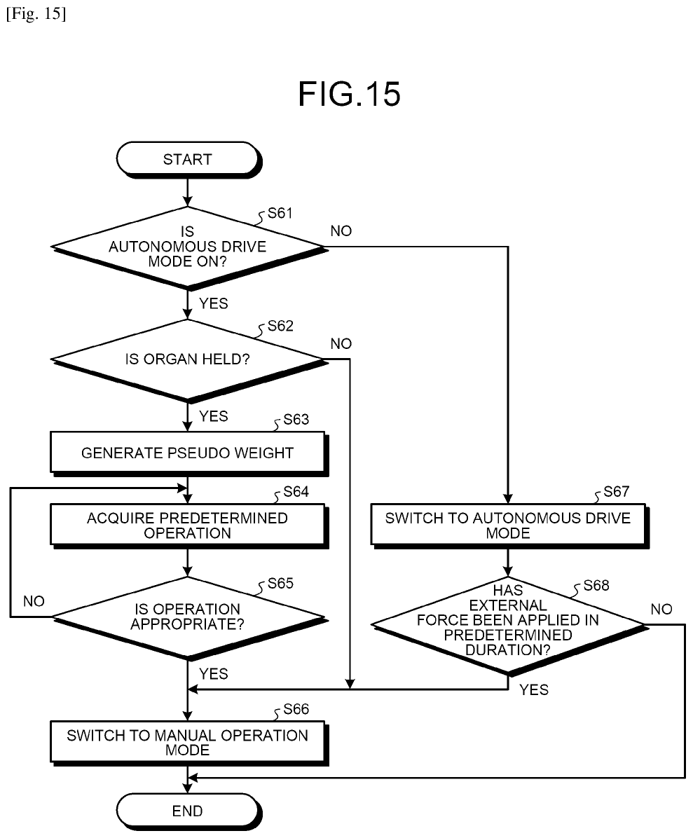

The following describes switching processing performed when the medical instrument mounted on the medical operation manipulator 300 is a retractor with reference to FIG. 15. FIG. 15 is a flowchart illustrating an exemplary process of switching processing performed when the medical instrument mounted on the medical operation manipulator 300 is a retractor.

Step S61 is the same as step S31 illustrated in FIG. 12, and thus description thereof is omitted.

When positive determination is made at step S61, the control unit 130 determines whether the retractor holds an organ (step S62). Specifically, the determination unit 133 determines whether the retractor holds an organ based on image data of the operation site acquired by the acquisition unit 131. When the retractor is provided with a pressure sensor, the determination unit 133 may determine whether the retractor holds an organ based on a measurement value obtained by the pressure sensor. When a pressure sensor is provided to a joint part of an arm device supporting the retractor, the determination unit 133 may determine that the retractor holds an organ when a value obtained by the pressure sensor is equal to or larger than the weight of the retractor. When it is determined that the retractor holds an organ (Yes at step S62), the process proceeds to step S63. When it is determined that the retractor does not hold an organ (No at step S62), the process proceeds to step S66.

When positive determination is made at step S62, the control unit 130 generates pseudo weight while the organ is held by the medical operation manipulator 300, (step S63). Specifically, for example, the manipulator control unit 135 generates pseudo weight by controlling the actuators at the joint parts of the support arm devices of the medical operation manipulator 300 and provides, to the operator, a load felt when switching is performed to the manual operation. In other words, when switching is performed to the manual operation, the operator is caused to execute a holding operation. Accordingly, it is possible to prevent a false operation due to abrupt application of the weight of the organ to the operator when switching to the manual operation. As a result, improved safety can be achieved. Then, the process proceeds to step S64.

The control unit 130 acquires a predetermined operation on the medical operation manipulator 300 (step S64). Specifically, the acquisition unit 131 acquires operation information related to the predetermined operation on the medical operation manipulator 300. Then, the process proceeds to step S65.

The control unit 130 determines whether the predetermined operation on the medical operation manipulator 300 is appropriate (step S65). Specifically, the determination unit 133 determines, based on the operation information acquired by the acquisition unit 131, whether the operator can reliably hold the retractor when switching is performed to the manual operation mode. For example, the determination unit 133 determines whether the medical operation manipulator 300 generating the pseudo weight is supported. When it is determined that the operation is appropriate (Yes at step S65), the process proceeds to step S66. When it is determined that the operation is inappropriate (No at step S65), the process proceeds to step S64. In other words, when it is determined that the operation is inappropriate, the processing at steps S63 and S64 is repeated until the operator performs an appropriate operation.

When positive determination is made at step S65, the control unit 130 switches the medical operation manipulator 300 to the manual operation mode (step S66). Specifically, the manipulator control unit 135 switches the medical operation manipulator 300 to the manual operation mode. In this case, instead of instantaneously canceling a holding force and performing switching to the manual operation, the manipulator control unit 135 gradually cancels the holding force and performs switching to the manual operation in a predetermined time. Accordingly, a load of the organ (or load, the scale of which is adjusted) is not applied to the operator at once when switching is performed to the manual operation mode, and thus switching can be securely performed to the manual operation while the organ is held. Then, the processing illustrated in FIG. 15 ends.

Steps S67 and S68 are the same as steps S36 and S37 illustrated in FIG. 12, respectively, and thus description thereof is omitted.

As described above, in the present embodiment, switching sequences can be changed in accordance with the medical instrument mounted on the medical operation manipulator 300. Accordingly, in the present embodiment, the autonomous drive mode and the manual operation mode can be more appropriately switched.

(4-3. Third Processing)

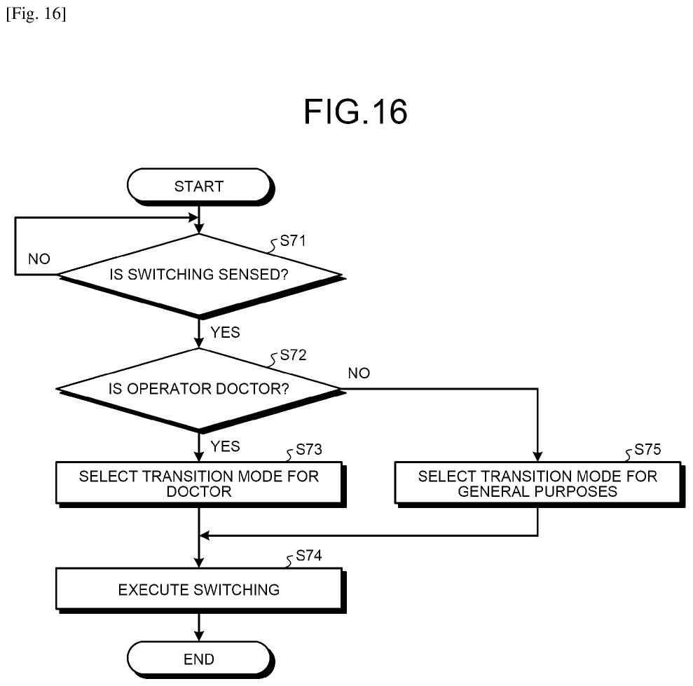

The following describes third processing performed at the control unit 130 of the control device 100 according to the embodiment of the present disclosure with reference to FIG. 16. FIG. 16 is a flowchart illustrating the process of the third processing performed at the control unit 130 of the control device 100 according to the embodiment of the present disclosure.

In accordance with whether the operator of the medical operation manipulator 300 is a doctor, changing a switching sequence through which the autonomous drive mode and the manual operation mode are switched is assumed to be preferable. Thus, the third processing executes processing of selecting a transition mode in accordance with the operator of the medical operation manipulator 300 or the attribute of the operator. In the third processing, the autonomous drive mode and the manual operation mode are switched through a switching sequence that differs in accordance with the operator of the medical operation manipulator 300.

Step S71 is the same as step S11 illustrated in FIG. 10, and thus description thereof is omitted.

When positive determination is made at step S71, the control unit 130 determines whether the operator of the medical operation manipulator 300 is a doctor (step S72). Specifically, the determination unit 133 determines whether the operator of the medical operation manipulator 300 is a doctor based on image data of the operator of the medical operation manipulator 300 acquired by the acquisition unit 131. Alternatively, the determination unit 133 may determine whether the operator is a doctor based on the biological information of the operator. For example, the determination unit 133 may determine whether the operator is a doctor by executing fingerprint authentication based on acquired fingerprint information of the operator and fingerprint information registered in advance. The determination unit 133 may determine various attributes of the operator of the medical operation manipulator 300. The determination unit 133 determines whether the operator of the medical operation manipulator 300 is a doctor based on, for example, information stored in the user information storage unit 121. When it is determined that the operator of the medical operation manipulator 300 is a doctor (Yes at step S72), the process proceeds to step S73. When it is determined that the operator of the medical operation manipulator 300 is not a doctor (No at step S72), the process proceeds to step S75.

When positive determination is made at step S72, the control unit 130 selects a transition mode of the medical operation manipulator 300 for doctor (step S73). Specifically, the selection unit 134 selects the transition mode for doctor based on a result of the determination by the determination unit 133. Then, the process proceeds to step S74.

The control unit 130 switches the autonomous drive mode and the manual operation mode of the medical operation manipulator 300 (step S74). Specifically, the manipulator control unit 135 switches the autonomous drive mode and the manual operation mode based on a result of the selection by the selection unit 134. Then, the processing illustrated in FIG. 16 ends.

When negative determination is made at step S72, the control unit 130 selects a transition mode of the medical operation manipulator 300 for general purposes (step S75). Specifically, the selection unit 134 selects the transition mode for general purposes based on the result of the determination by the determination unit 133. Then, the process proceeds to step S74.

In the transition mode for general purposes, for example, a confirmation operation on whether to execute switching is executed. In this case, switching is executed only when confirmation is input. When a person having no medical license operates the medical operation manipulator 300, a transition mode in which a manual operation can be performed only in the direction of retraction from the patient or the treatment site may be executed.

As described above, in the present embodiment, switching sequences can be changed in accordance with the operator of the medical operation manipulator 300. Accordingly, in the present embodiment, the autonomous drive mode and the manual operation mode can be more appropriately switched.

(4-4. Fourth Processing)

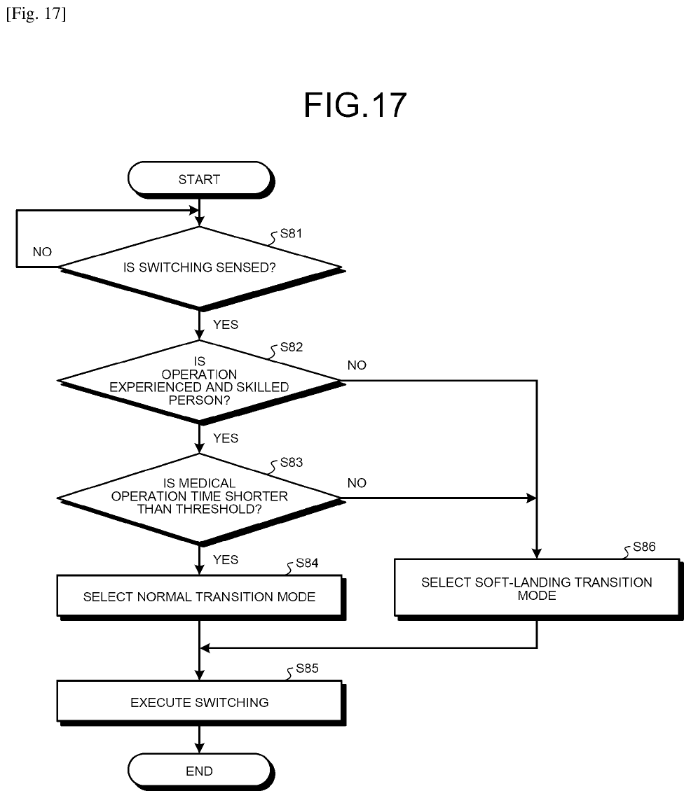

The following describes fourth processing performed at the control unit 130 of the control device 100 according to the embodiment of the present disclosure with reference to FIG. 17. FIG. 17 is a flowchart illustrating the process of the fourth processing performed at the control unit 130 of the control device 100 according to the embodiment of the present disclosure.

In accordance with the skill and medical operation time of the operator of the medical operation manipulator 300, changing a switching sequence through which the autonomous drive mode and the manual operation mode are switched is assumed to be preferable. Thus, the fourth processing executes processing of selecting a transition mode in accordance with the profile and medical operation time of the operator of the medical operation manipulator 300. In the fourth processing, the autonomous drive mode and the manual operation mode are switched through a switching sequence that differs in accordance with the profile and medical operation time of the operator of the medical operation manipulator 300.

Step S81 is the same as step S11 illustrated in FIG. 10, and thus description thereof is omitted.

When positive determination is made at step S81, the control unit 130 determines whether the operator of the medical operation manipulator 300 is an experienced and skilled person (step S82). Specifically, the determination unit 133 determines whether the operator of the medical operation manipulator 300 is an experienced and skilled person based on image data of the operator of the medical operation manipulator 300 acquired by the acquisition unit 131. The determination unit 133 determines whether the operator of the medical operation manipulator 300 is a veteran doctor based on, for example, information stored in the user information storage unit 121. Alternatively, the determination unit 133 may determine whether the operator is experienced and skilled person based on the medical operation skill of the operator of the medical operation manipulator 300. More specifically, the determination unit 133 determines a veteran doctor with sufficient medical operation experience, a doctor having a high medical operation skill, and the like to be an experienced and skilled person. Alternatively, the determination unit 133 may determine whether the operator is an experienced and skilled person based on the biological information of the operator. When it is determined that the operator of the medical operation manipulator 300 is an experienced and skilled person (Yes at step S82), the process proceeds to step S83. When it is determined that the operator of the medical operation manipulator 300 is not an experienced and skilled person (No at step S82), the process proceeds to step S86.

When positive determination is made at step S82, the control unit 130 determines whether the medical operation time is shorter than a threshold (step S83). Specifically, the determination unit 133 determines whether the medical operation time is shorter than the threshold based on information stored in the medical operation information storage unit 122. When it is determined that the medical operation time is shorter than the threshold (Yes at step S83), the process proceeds to step S84. When it is determined that the medical operation time is longer than the threshold (No at step S83), the process proceeds to step S86.

When positive determination is made at step S83, the control unit 130 selects a normal transition mode of the medical operation manipulator 300 (step S84). Specifically, the selection unit 134 selects the normal transition mode based on a result of the determination by the determination unit 133. Then, the process proceeds to step S85.

The control unit 130 switches the autonomous drive mode and the manual operation mode of the medical operation manipulator 300 (step S85). Specifically, the manipulator control unit 135 switches the autonomous drive mode and the manual operation mode based on a result of the selection by the selection unit 134. Then, the processing illustrated in FIG. 17 ends.

When negative determination is made at step S82 or S83, the control unit 130 selects a soft-landing transition mode of the medical operation manipulator 300 (step S86). Specifically, the selection unit 134 selects the soft-landing transition mode based on the result of the determination by the determination unit 133. In the soft-landing transition mode, a time taken for transition is set to be longer than in the normal transition mode. In other words, in the present embodiment, the switching operation is executed over a longer time when the operator is a less experienced doctor or when the degree of fatigue of the doctor is assumed to be high due to a long medical operation. Accordingly, improved safety can be achieved.

As described above, in the present embodiment, switching sequences can be changed in accordance with the skill of the operator of the medical operation manipulator 300 and the degree of fatigue of the operation in a medical operation. Accordingly, in the present embodiment, the autonomous drive mode and the manual operation mode can be more appropriately switched.

(4-5. Fifth Processing)

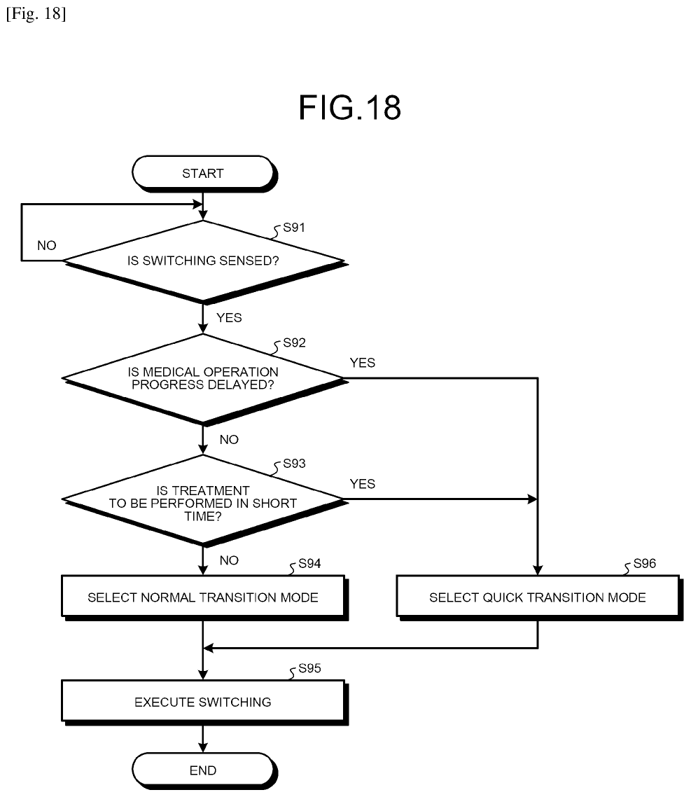

The following describes fifth processing performed at the control unit 130 of the control device 100 according to the embodiment of the present disclosure with reference to FIG. 18. FIG. 18 is a flowchart illustrating the process of the fifth processing performed at the control unit 130 of the control device 100 according to the embodiment of the present disclosure.

In accordance with the degree of progress of a medical operation and the kind of processing with the medical operation manipulator 300, changing a switching sequence through which the autonomous drive mode and the manual operation mode are switched is assumed to be preferable. Thus, the fifth processing executes processing of selecting a transition mode in accordance with the degree of progress of the medical operation and the kind of treatment. For example, in the fifth processing, a transition mode is selected from among a plurality of transition modes having transition times different from each other in accordance with the status of the medical operation. In other words, in the fifth processing, the autonomous drive mode and the manual operation mode are switched through a switching sequence that differs in accordance with the degree of progress of the medical operation and the kind of treatment.

Step S91 is the same as step S11 illustrated in FIG. 10, and thus description thereof is omitted.

When positive determination is made at step S91, the control unit 130 determines whether the progress of the medical operation is delayed (step S92). Specifically, the determination unit 133 determines whether the progress of the medical operation is delayed based on information related to the medical operation time stored in the medical operation information storage unit 122. When it is determined that the progress of the medical operation is not delayed (No at step S92), the process proceeds to step S93. When it is determined that the progress of the medical operation is delayed (Yes at step S92), the process proceeds to step S96.

When negative determination is made at step S92, the control unit 130 determines whether currently performed treatment is to be performed in a short time (step S93). Specifically, the determination unit 133 determines whether the currently performed treatment is to be performed in a short time based on image data of the treatment acquired by the acquisition unit 131 and information stored in the medical operation information storage unit 122. When it is determined that the currently performed is not to be performed in a short time (No at step S93), the process proceeds to step S94. When it is determined that the currently performed treatment is to be performed in a short time (Yes at step S93), the process proceeds to step S96.

When negative determination is made at step S93, the control unit 130 selects the normal transition mode of the medical operation manipulator 300 (step S94). Specifically, the selection unit 134 selects the normal transition mode based on the result of the determination by the determination unit 133. Then, the process proceeds to step S95.

The control unit 130 switches the autonomous drive mode and the manual operation mode of the medical operation manipulator 300 (step S95). Specifically, the manipulator control unit 135 switches the autonomous drive mode and the manual operation mode based on a result of the selection by the selection unit 134. Then, the processing illustrated in FIG. 18 ends.

When positive determination is made at step S92 or S93, the control unit 130 selects a quick transition mode of the medical operation manipulator 300 (step S96). Specifically, the selection unit 134 selects the quick transition mode based on the result of the determination by the determination unit 133. In the quick transition mode, a time taken for transition is set to be shorter than in the normal transition mode. In other words, in the present embodiment, the autonomous drive mode and the manual operation mode can be swiftly switched when the progress of the medical operation is delayed or when the treatment is to be performed in a short time.

As described above, in the present embodiment, switching sequences can be changed in accordance with the degree of progress of the medical operation and the kind of treatment with the medical operation manipulator 300. Accordingly, in the present embodiment, the autonomous drive mode and the manual operation mode can be more appropriately switched.

(4-6. Sixth Processing)

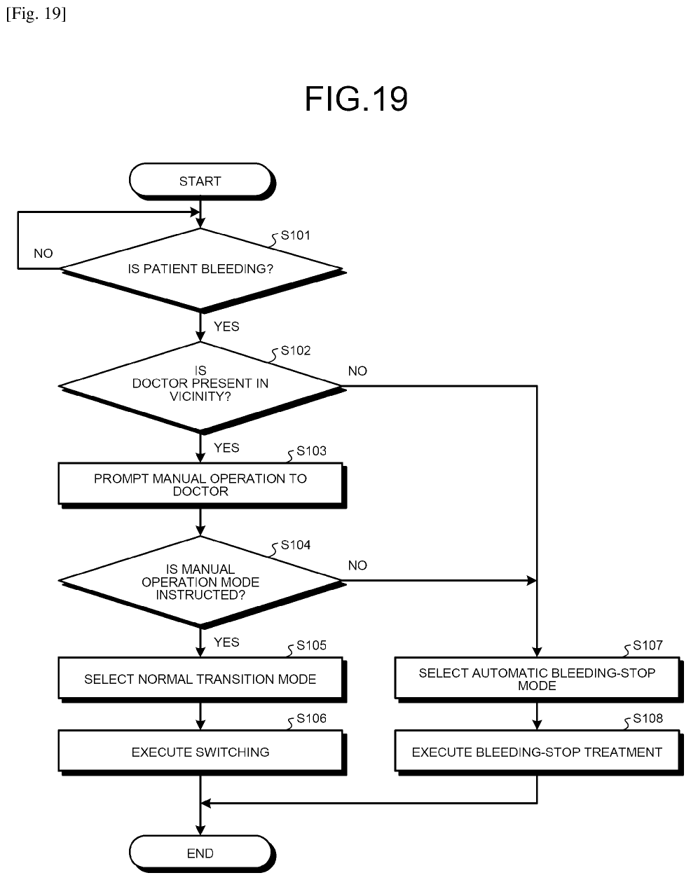

The following describes sixth processing performed at the control unit 130 of the control device 100 according to the embodiment of the present disclosure with reference to FIG. 19. FIG. 19 is a flowchart illustrating the process of the sixth processing performed at the control unit 130 of the control device 100 according to the embodiment of the present disclosure.

In accordance with whether a doctor is present in the vicinity of the medical operation manipulator 300, changing a switching sequence through which the autonomous drive mode and the manual operation mode are switched is assumed to be preferable. Thus, the sixth processing executes processing of selecting a transition mode in accordance with whether a doctor is present in the vicinity of the medical operation manipulator 300. In the sixth processing, the autonomous drive mode and the manual operation mode are switched through a switching sequence that differs in accordance with whether a doctor is present in the vicinity of the medical operation manipulator 300.

The control unit 130 determines whether the patient is bleeding (step S101). Specifically, the determination unit 133 determines whether the patient is bleeding based on image data of the operation site acquired by the acquisition unit 131. When it is determined that the patient is bleeding (Yes at step S101), the process proceeds to step S102. When it is determined that the patient is not bleeding (No at step S101), the processing at step S101 is repeated.

When positive determination is made at step S101, the control unit 130 determines whether a doctor is present in the vicinity of the medical operation manipulator 300 (step S102). Specifically, the determination unit 133 determines whether a doctor is present in the vicinity of the medical operation manipulator 300 based on image data of the vicinity of the medical operation manipulator 300 acquired by the acquisition unit 131. Alternatively, for example, the determination unit 133 may acquire, through the medical operation manipulator 300, the biological information of any person in the vicinity of the medical operation manipulator 300, and may determine whether the person is a doctor based on the acquired biological information. The vicinity of the medical operation manipulator 300 means, for example, a distance with which a doctor can apply external force to the medical operation manipulator 300 to execute an action to prevent danger. The determination unit 133 determines whether the person in the vicinity of the medical operation manipulator 300 is a doctor based on, for example, information stored in the user information storage unit 121. When it is determined that a doctor is present in the vicinity of the medical operation manipulator 300 (Yes at step S102), the process proceeds to step S103. When it is determined that no doctor is present in the vicinity of the medical operation manipulator 300 (No at step S102), the process proceeds to step S107.

When positive determination is made at step S102, the control unit 130 prompts the doctor to perform a manual operation (step S103). Specifically, the manipulator control unit 135 drives the medical operation manipulator 300 to prompt the doctor to perform the manual operation. The control unit 130 may prompt the doctor to perform the manual operation by outputting voice from a speaker or displaying an alert image on a display unit. Then, the process proceeds to step S104.

It is determined whether the doctor instructs the manual operation mode (step S104). Specifically, the determination unit 133 determines whether the doctor instructs the manual operation mode based on the operation information acquired by the acquisition unit 131. When the doctor instructs the manual operation mode (Yes at step S104), the process proceeds to step S105. When the doctor instructs the autonomous drive mode (No at step S104), the process proceeds to step S107.

When positive determination is made at step S104, the control unit 130 selects the normal transition mode of the medical operation manipulator 300 (step S105). Specifically, the selection unit 134 selects the normal transition mode based on the result of the determination by the determination unit 133. Then, the process proceeds to step S106.

The control unit 130 switches the medical operation manipulator 300 to the manual operation mode (step S106). Specifically, the manipulator control unit 135 performs switching to the manual operation mode based on a result of the selection by the selection unit 134. Then, the processing illustrated in FIG. 19 ends.

When negative determination is made at step S102 or S104, the control unit 130 selects an automatic bleeding-stop mode of the medical operation manipulator 300 (step S107). Specifically, the selection unit 134 selects the automatic bleeding-stop mode based on the result of the determination by the determination unit 133. In the automatic bleeding-stop mode, the medical operation manipulator 300 is autonomously driven to perform treatment on an affected part to stop bleeding. Then, the process proceeds to step S108.

The control unit 130 causes the medical operation manipulator 300 to execute treatment on the affected part to stop bleeding (step S108). Specifically, the manipulator control unit 135 causes the medical operation manipulator 300 to execute treatment on the affected part to stop bleeding by controlling the medical operation manipulator 300. Then, the processing illustrated in FIG. 19 ends.

As described above, in the present embodiment, it is determined whether a doctor is present in the vicinity of the medical operation manipulator 300. When a doctor is present, the autonomous drive mode and the manual operation mode are switched in accordance with an instruction from the doctor. When no doctor is present, switching is performed to the autonomous drive mode. Accordingly, in the present embodiment, the autonomous drive mode and the manual operation mode can be more appropriately switched.

(4-7. Seventh Processing)

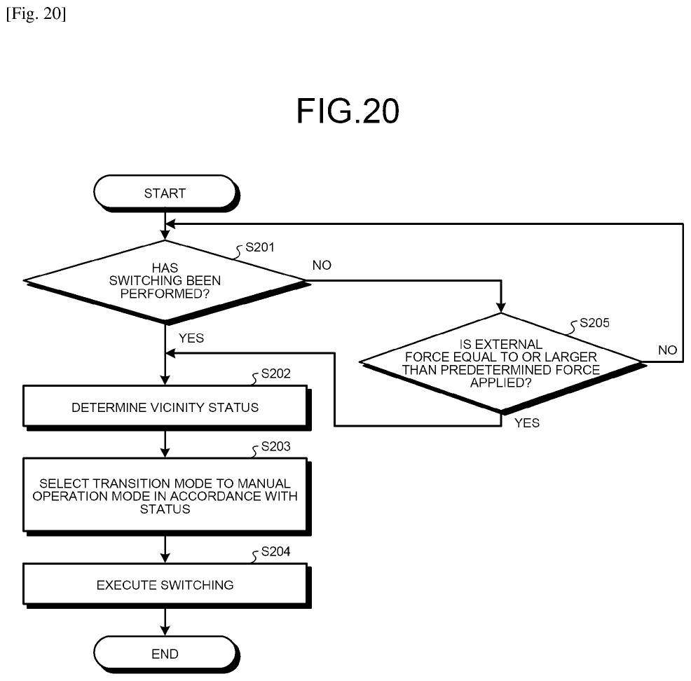

The following describes seventh processing performed at the control unit 130 of the control device 100 according to the embodiment of the present disclosure with reference to FIG. 20. FIG. 20 is a flowchart illustrating the process of the seventh processing performed at the control unit 130 of the control device 100 according to the embodiment of the present disclosure.

In accordance with whether external force is applied to the medical operation manipulator 300, changing a switching sequence through which switching is performed from the autonomous drive mode to the manual operation mode is assumed to be preferable. Thus, the seventh processing executes processing of performing switching from the autonomous drive mode to the manual operation mode when external force is applied to the medical operation manipulator 300. In the seventh processing, the autonomous drive mode and the manual operation mode are switched through a switching sequence that differs in accordance with the magnitude of the external force applied to the medical operation manipulator 300.

Steps S201, S202, 5203, and 5204 are the same as steps S11, S13, S14, and S15 illustrated in FIG. 10, respectively, and thus description thereof is omitted.

When negative determination is made at step S201, the control unit 130 determines whether external force equal to or larger than a predetermined force is applied to the medical operation manipulator 300 (step S205). Specifically, the determination unit 133 determines whether the external force equal to or larger than the predetermined force is applied to the medical operation manipulator 300 based on external force information acquired by the acquisition unit 131. The external force equal to or larger than the predetermined force may be force with which it is determined that the operator intends to stop drive of the autonomously driven medical operation manipulator 300. The determination unit 133 may determine the status of the medical operation and change, in accordance with the status of the medical operation, a threshold based on which it is determined whether the external force is equal to or larger than the predetermined force. When it is determined that no external force equal to or larger than the predetermined force is applied to the medical operation manipulator 300 (No at step S205), the process proceeds to step S201. When it is determined that the external force equal to or larger than the predetermined force is applied to the medical operation manipulator 300 (Yes at step S205), the process proceeds to step S202. In other words, when external force equal to or larger than the predetermined force is applied to the medical operation manipulator 300 although no switching of the medical operation manipulator 300 is sensed, intervention from the operator is determined and switching is performed to the manual operation mode.

As described above, in the present embodiment, switching can be performed from the autonomous drive mode to the manual operation mode when external force is applied to the medical operation manipulator 300. Accordingly, in the present embodiment, for example, when the medical operation manipulator 300 operates in a manner different from determination by a doctor, switching can be performed to the manual operation mode by applying external force to the medical operation manipulator 300, which leads to improved safety.

(4-8. Eighth Processing)

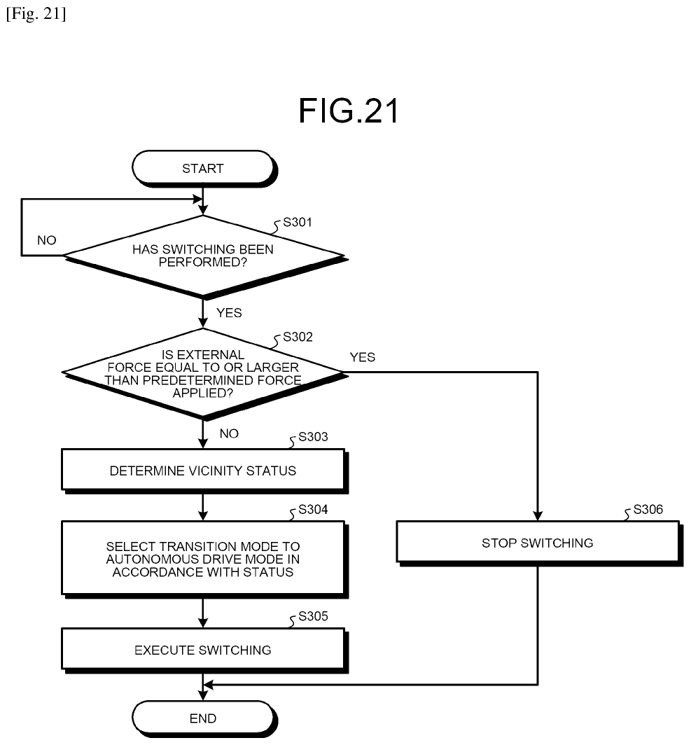

The following describes eighth processing performed at the control unit 130 of the control device 100 according to the embodiment of the present disclosure with reference to FIG. 21. FIG. 21 is a flowchart illustrating the process of the eighth processing performed at the control unit 130 of the control device 100 according to the embodiment of the present disclosure.

In accordance with whether external force is applied to the medical operation manipulator 300, changing a switching sequence through which switching is performed from the manual operation mode to the autonomous drive mode is assumed to be preferable. Thus, the eighth processing executes processing of performing switching from the manual operation mode to the autonomous drive mode when external force is applied to the medical operation manipulator 300. In the eighth processing, the autonomous drive mode and the manual operation mode are switched through a switching sequence that differs in accordance with the magnitude of the external force applied to the medical operation manipulator 300.

Step S301 is the same as step S11 illustrated in FIG. 10, and thus description thereof is omitted.

When negative determination is made at step S301, the control unit 130 determines whether external force equal to or larger than the predetermined force is applied to the medical operation manipulator 300 (step S302). Specifically, the determination unit 133 determines whether the external force equal to or larger than the predetermined force is applied based on external force information acquired by the acquisition unit 131. When it is determined that no external force equal to or larger than the predetermined force is applied to the medical operation manipulator 300 (No at step S302), the process proceeds to step S303. When it is determined that the external force equal to or larger than the predetermined force is applied to the medical operation manipulator 300 (Yes at step S302), the process proceeds to step S306.

Steps S303, S304, and S305 are the same as steps S16, S17, and S15 illustrated in FIG. 10, respectively, and thus description thereof is omitted.

When positive determination is made at step S302, the control unit 130 stops switching of the medical operation manipulator 300 (step S306). Specifically, the manipulator control unit 135 stops switching of the medical operation manipulator 300. In other words, in the present embodiment, when the external force equal to or larger than the predetermined force is applied to the medical operation manipulator 300 while switching of the medical operation manipulator 300 is sensed, intervention from the operator is determined and the manual operation mode is maintained.

As described above, in the present embodiment, switching to the autonomous drive mode can be stopped when external force is applied to the medical operation manipulator 300. Accordingly, in the present embodiment, improved safety is achieved.

(4-9. Ninth Processing)

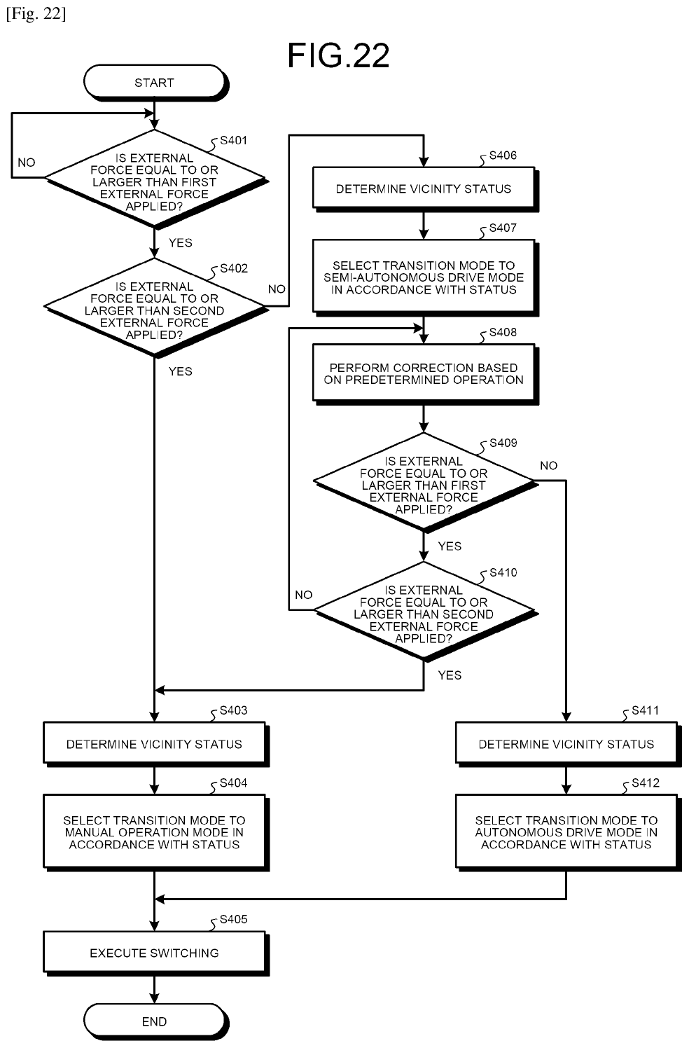

The following describes ninth processing performed at the control unit 130 of the control device 100 according to the embodiment of the present disclosure with reference to FIG. 22. FIG. 22 is a flowchart illustrating the process of the ninth processing performed at the control unit 130 of the control device 100 according to the embodiment of the present disclosure.

When the medical operation manipulator 300 is autonomously driven, the operation of the medical operation manipulator 300 is different from that expected by a doctor although intervention is not required, and thus correction of the operation is assumed. In the ninth processing, a first external force and a second external force larger than the first external force are set, and processing of performing switching from the autonomous drive mode to the manual operation mode is changed in accordance with the magnitude of applied external force. In the ninth processing, the autonomous drive mode and the manual operation mode are switched through a switching sequence that differs in accordance with the kind and magnitude of the external force applied to the medical operation manipulator 300. The second external force is an external force beyond which intervention from the operator is determined.

The control unit 130 determines whether external force equal to or larger than the first external force is applied to the medical operation manipulator 300 (step S401). Specifically, the determination unit 133 determines whether the external force equal to or larger than the first external force is applied to the medical operation manipulator 300 based on external force information acquired by the acquisition unit 131. When it is determined that no external force equal to or larger than the first external force is applied to the medical operation manipulator 300 (No at step S401), the processing at step S401 is repeated. When it is determined that the external force equal to or larger than the first external force is applied to the medical operation manipulator 300 (Yes at step S401), the process proceeds to step S402.

When positive determination is made at step S401, the control unit 130 determines whether external force equal to or larger than the second external force is applied to the medical operation manipulator 300 (step S402). Specifically, the determination unit 133 determines whether the external force equal to or larger than the second external force is applied to the medical operation manipulator 300 based on external force information acquired by the acquisition unit 131. When it is determined that the external force equal to or larger than the second external force is applied to the medical operation manipulator 300 (Yes at step S402), the process proceeds to step S403. When it is determined that no external force equal to or larger than the second external force is applied to the medical operation manipulator 300 (No at step S402), the process proceeds to step S406.

Steps S403 to S405 are the same as steps S13 to S15 illustrated in FIG. 10, respectively, and thus description thereof is omitted.

When negative determination is made at step S402, the control unit 130 determines the status of the vicinity of the medical operation manipulator 300 (step S406). This processing is the same as that at step S13 illustrated in FIG. 10, and thus description thereof is omitted. Then, the process proceeds to step S407.

The control unit 130 selects a transition mode in which transition is performed from the autonomous drive mode to a semi-autonomous drive mode in accordance with the status of the vicinity of the medical operation manipulator 300 (step S407). In the semi-autonomous drive mode, autonomous drive is corrected based on an operation from the operator. In other words, in the semi-autonomous drive mode, the medical operation manipulator 300 executes autonomous drive. Specifically, the determination unit 133 determines that operation correction is to be performed on the medical operation manipulator 300 because the first external force is applied to the medical operation manipulator 300. Then, the selection unit 134 selects the semi-autonomous drive mode to perform the operation correction. Then, the process proceeds to step S408.

The control unit 130 corrects the operation of the medical operation manipulator 300 based on a predetermined operation (step S408). Specifically, the acquisition unit 131 acquires operation information related to operation correction on the medical operation manipulator 300 with force equal to or larger than the first external force and smaller than the second external force. Then, the manipulator control unit 135 corrects the operation of the medical operation manipulator 300. Then, the process proceeds to step S409.

The control unit 130 determines whether external force equal to or larger than the first external force is applied to the medical operation manipulator 300 (step S409). When it is determined that the external force equal to or larger than the first external force is applied to the medical operation manipulator 300 (Yes at step S409), the process proceeds to step S410. When it is determined that no force equal to or larger than the first external force is applied to the medical operation manipulator 300 (No at step S409), the process proceeds to step S411.

When positive determination is made at step S409, the control unit 130 determines whether external force equal to or larger than the second external force is applied to the medical operation manipulator 300 (step S410). When it is determined that the force equal to or larger than the second external force is applied to the medical operation manipulator 300 (Yes at step S410), the process proceeds to step S403. In this case, intervention from the operator is determined, and switching is performed from the autonomous drive mode to the manual operation mode. When it is determined no force equal to or larger than the second external force is applied to the medical operation manipulator 300 (No at step S410), the process proceeds to step S408. In this case, when force equal to or larger than the first external force is determined but it is determined that no external force equal to or larger than the second external force is applied, the semi-autonomous drive mode is continued.

When negative determination is made at step S409, the control unit 130 determines the status of the vicinity of the medical operation manipulator 300 (step S411). This processing is the same as that at step S13 illustrated in FIG. 10, and thus description thereof is omitted. Then, the process proceeds to step S412.

The control unit 130 selects a transition mode in which transition is performed from the semi-autonomous drive mode to the autonomous drive mode in accordance with the status of the vicinity of the medical operation manipulator 300 (step S412). Specifically, the determination unit 133 determines that the operation correction on the medical operation manipulator 300 has ended because the first external force is not applied to the medical operation manipulator 300. Then, the selection unit 134 selects the autonomous drive mode. Then, the processing illustrated in FIG. 22 ends.

As described above, in the present embodiment, for example, when the operation of the medical operation manipulator 300 being autonomously driven is different from that expected by a doctor, switching can be performed to the semi-autonomous drive mode so that the operation of the medical operation manipulator 300 in the autonomous drive can be corrected. Accordingly, improved safety is achieved for a medical operation.

### 5. Hardware Configuration

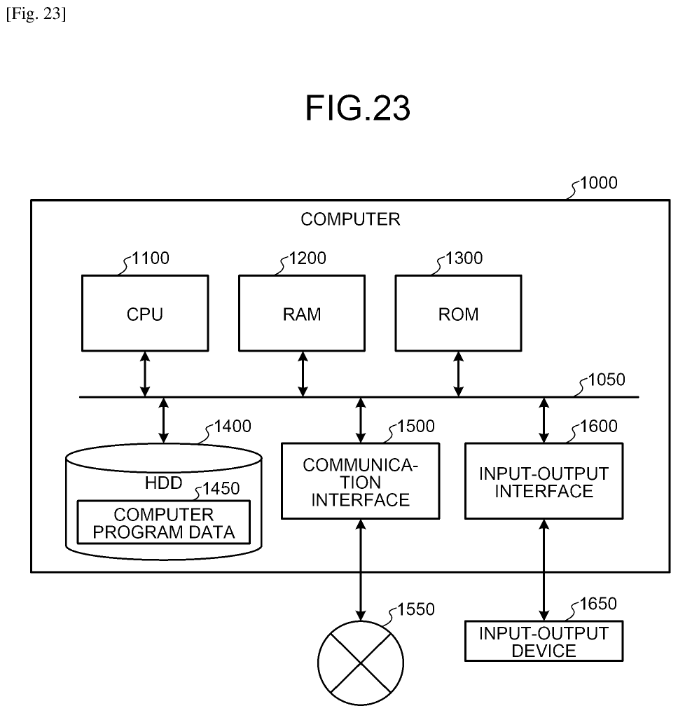

An information instrument such as the control device 100 described above is achieved by, for example, a computer 1000 having a configuration as illustrated in FIG. 23. FIG. 23 is a hardware configuration diagram illustrating an exemplary computer 1000 configured to achieve functions of an information processing device such as the control device 100. The following exemplarily describes the control device 100 according to the embodiment. The computer 1000 includes a CPU 1100, a RAM 1200, a read only memory (ROM) 1300, a hard disk drive (HDD) 1400, a communication interface 1500, and an input-output interface 1600. The components of the computer 1000 are connected with each other through a bus 1050.

The CPU 1100 operates based on computer programs stored in the ROM 1300 or the HDD 1400 and controls each component. For example, the CPU 1100 loads the computer programs stored in the ROM 1300 or the HDD 1400 onto the RAM 1200 and executes various kinds of processing corresponding to the computer programs.

The ROM 1300 stores a boot program such as a basic input output system (BIOS) executed by the CPU 1100 at activation of the computer 1000, and a computer program dependent on the hardware of the computer 1000, and the like.

the HDD 1400 is a computer-readable recording medium non-temporarily recording a computer program executed by the CPU 1100, data used by the computer program, and the like. Specifically, the HDD 1400 is a recording medium recording the information processing program according to the present disclosure as exemplary computer program data 1450.

The communication interface 1500 is an interface through which the computer 1000 is connected with an external network 1550 (for example, the Internet). For example, through the communication interface 1500, the CPU 1100 receives data from another device and transmits data generated by the CPU 1100 to another device.

The input-output interface 1600 is an interface through which the computer 1000 is connected with an input-output device 1650. For example, the CPU 1100 receives data from an input device such as a keyboard or a mouse through the input-output interface 1600. In addition, the CPU 1100 transmits data to an output device such as a display, a speaker, or a printer through the input-output interface 1600. The input-output interface 1600 may function as a media interface through which a computer program or the like recorded in a predetermined recording medium is read. The medium is, for example, an optical recording medium such as a digital versatile disc (DVD) or a phase change rewritable disk (PD), a magneto-optical recording medium such as a magneto-optical disk (MO), a tape medium, a magnetic recording medium, or a semiconductor memory.

For example, when the computer 1000 functions as the control device 100 according to the embodiment, the CPU 1100 of the computer 1000 achieves the function of the control unit 130 or the like by executing the information processing program loaded onto the RAM 1200. The HDD 1400 stores the information processing program according to the present disclosure and data in a storage unit 14. Although the CPU 1100 reads the computer program data 1450 from the HDD 1400 for execution, the computer program may be acquired from another device through the external network 1550, as another example.

(Effects)

The medical operation support system 1 includes: the medical operation manipulator 300; the sensing unit 132 configured to sense a switching operation that performs switching between the autonomous drive mode and the manual operation mode of the medical operation manipulator 300; the determination unit 133 configured to determine the status of the medical operation room in which the medical operation manipulator 300 is disposed when the switching operation is sensed by the sensing unit 132; and the selection unit 134 configured to select a switching sequence in accordance with the status determined by the determination unit 133.

With this configuration, an appropriate sequence through which the autonomous drive mode and the manual operation mode are switched can be selected in accordance with the status of the medical operation room. As a result, the autonomous drive and the manual operation of the medical operation manipulator 300 can be securely switched.

The determination unit 133 may determine the state of the medical operation manipulator 300, and the selection unit 134 may select a switching sequence in accordance with the state of the medical operation manipulator 300.

With this configuration, an appropriate sequence through which the autonomous drive and the manual operation are switched can be selected in accordance with the state of the medical operation manipulator 300. As a result, the autonomous drive mode and the manual operation mode of the medical operation manipulator 300 can be securely switched.

The determination unit 133 may determine whether the medical operation manipulator 300 is in the autonomous drive mode or the manual operation mode.

With this configuration, whether the medical operation manipulator 300 is in the autonomous drive mode or the manual operation mode can be determined. As a result, a switching sequence can be selected in accordance with whether the medical operation manipulator 300 is in the autonomous drive mode or the manual operation mode.

The determination unit 133 may determine a medical instrument mounted on the medical operation manipulator 300, and the selection unit 134 may select a switching sequence in accordance with the medical instrument mounted on the medical operation manipulator 300.

With this configuration, an appropriate sequence can be selected in accordance with the kind of the medical instrument mounted on the medical operation manipulator 300. As a result, the autonomous drive and the manual operation of the medical operation manipulator 300 can be securely switched.

The determination unit 133 may determine whether the medical operation manipulator 300 is in the autonomous drive mode or the manual operation mode, and the selection unit 134 may select a switching sequence in accordance with the medical instrument mounted on the medical operation manipulator 300 and whether the medical operation manipulator 300 is in the autonomous drive mode or the manual operation mode.

With this configuration, an appropriate sequence can be selected in accordance with the kind of the medical instrument mounted on the medical operation manipulator 300 and a mode. As a result, the autonomous drive and the manual operation of the medical operation manipulator 300 can be securely switched.

When it is determined that the medical instrument is a camera or a scalpel and when the medical operation manipulator 300 is in the autonomous drive mode, the selection unit 134 may select a switching sequence through which switching is performed to the manual operation mode in stages.

With this configuration, when the medical instrument is a camera or a scalpel, switching can be performed from the autonomous drive mode to the manual operation mode in stages. As a result, the autonomous drive and the manual operation of the medical operation manipulator 300 can be securely switched.

When it is determined that the medical instrument is a needle holder and when the medical operation manipulator 300 is in the autonomous drive mode and a needle grasped by the needle holder is inserted into an affected part, the selection unit 134 may select a switching sequence through which switching is performed to the manual operation mode in stages.

With this configuration, when the medical instrument is a needle holder and a needle grasped by the needle holder is inserted into an affected part, switching can be performed from the autonomous drive mode to the manual operation mode in stages. As a result, the autonomous drive and the manual operation of the medical operation manipulator 300 can be securely switched.

When it is determined that the medical instrument is a retractor and when the medical operation manipulator 300 is in the autonomous drive mode and the retractor holds an organ, the selection unit 134 may select a switching sequence through which pseudo weight is generated at the medical operation manipulator 300.

With this configuration, when the medical instrument is a retractor and the retractor holds an organ, switching can be performed from the autonomous drive mode to the manual operation mode in stages after pseudo weight is generated. As a result, the autonomous drive and the manual operation of the medical operation manipulator 300 can be securely switched.

After generating pseudo weight at the medical operation manipulator 300, the selection unit 134 may select a switching sequence through which switching is performed to the manual operation mode when a predetermined operation is received.

With this configuration, when the medical instrument is a retractor and the retractor holds an organ, switching can be performed from the autonomous drive mode to the manual operation mode in stages upon reception of predetermined operation after pseudo weight is generated. As a result, the autonomous drive and the manual operation of the medical operation manipulator 300 can be securely switched.

The medical operation manipulator 300 may include an arm unit (310, 320, 330) at least partially bendable and capable of supporting the medical instrument.

With this configuration, the present technology can be applied to the medical operation manipulator 300 including an arm unit. As a result, improved versatility can be achieved.

The determination unit 133 may determine the state of an operator having executed the switching operation on the medical operation manipulator 300, and the selection unit 134 may select a switching sequence in accordance with the state of the operator.

With this configuration, an appropriate sequence can be selected in accordance with the state of the operator of the medical operation manipulator 300. As a result, the autonomous drive mode and the manual operation mode of the medical operation manipulator 300 can be securely switched.

The determination unit 133 may determine the attribute of the operator, and the selection unit 134 may select a switching sequence in accordance with the attribute of the operator.

With this configuration, an appropriate sequence can be selected in accordance with the attribute of the operator of the medical operation manipulator 300. As a result, the autonomous drive mode and the manual operation mode of the medical operation manipulator 300 can be securely switched.

The determination unit 133 may determine whether the operator is a doctor, and the selection unit 134 may select a switching sequence in accordance with whether the operator is a doctor.

With this configuration, an appropriate sequence can be selected in accordance with whether the operator of the medical operation manipulator 300 is a doctor. Accordingly, an appropriate switching sequence can be selected in accordance with whether the operator is a doctor when the autonomous drive mode and the manual operation mode of the medical operation manipulator 300 are switched. As a result, the autonomous drive mode and the manual operation mode of the medical operation manipulator 300 can be securely switched.

The determination unit 133 may determine a record of biological information of the operator, and the selection unit 134 may select a switching sequence in accordance with the record of biological information of the operator.

With this configuration, an appropriate sequence can be selected in accordance with the biological information of the operator of the medical operation manipulator 300. Accordingly, an appropriate switching sequence can be selected in accordance with the degree of fatigue of the medical operation manipulator 300 or the like when the autonomous drive mode and the manual operation mode of the medical operation manipulator 300 are switched. As a result, the autonomous drive mode and the manual operation mode of the medical operation manipulator 300 can be securely switched.

The determination unit 133 may determine the status of a medical operation being performed in the medical operation room, and the selection unit 134 may select a switching sequence in accordance with the status of the medical operation being performed in the medical operation room.

With this configuration, an appropriate sequence can be selected in accordance with the status of the medical operation. As a result, the autonomous drive and the manual operation of the medical operation manipulator 300 can be securely switched.

The determination unit 133 may determine the degree of progress of the medical operation being performed in the medical operation room, and the selection unit 134 may select a switching sequence in accordance with the degree of progress of the medical operation being performed in the medical operation room.

With this configuration, an appropriate sequence can be selected in accordance with the degree of progress of the medical operation. Accordingly, an appropriate switching sequence can be selected when the autonomous drive mode and the manual operation mode of the medical operation manipulator 300 are switched while, for example, the medical operation is delayed than expected. As a result, the autonomous drive mode and the manual operation mode of the medical operation manipulator 300 can be securely switched.

The selection unit 134 may select a switching sequence having a different transition time in accordance with the degree of progress of the medical operation being performed in the medical operation room.

With this configuration, when the degree of progress of the medical operation is delayed and treatment is to be performed in a short time, a transition mode with a time shorter than normal can be selected. As a result, improved safety is achieved for a medical operation.

The determination unit 133 may determine existence of bleeding in an operative field, and the selection unit 134 may select a switching sequence in accordance with the existence of bleeding in the operative field.

With this configuration, an appropriate sequence can be selected in accordance with the existence of bleeding from the patient. Accordingly, an appropriate switching sequence can be selected when the autonomous drive mode and the manual operation mode of the medical operation manipulator 300 are switched while, for example, the patient is bleeding. As a result, the autonomous drive mode and the manual operation mode of the medical operation manipulator 300 can be securely switched.

The determination unit 133 may determine whether a doctor is present in the vicinity of the medical operation manipulator 300, and the selection unit 134 may select a switching sequence in accordance with whether a doctor is present in the vicinity of the medical operation manipulator 300.

With this configuration, different switching sequences can be selected in accordance with whether a doctor is present in the vicinity of the medical operation manipulator 300. As a result, the autonomous drive mode and the manual operation mode of the medical operation manipulator 300 can be securely switched.

The selection unit 134 may select a switching sequence through which the medical operation manipulator 300 is caused to stop bleeding in the operative field when it is determined that no doctor is present in the vicinity of the medical operation manipulator 300 and the medical operation manipulator 300 is in the autonomous drive mode.

With this configuration, when bleeding needs to be stopped but no doctor is present in the vicinity of the medical operation manipulator 300, a switching sequence through which the medical operation manipulator 300 is caused to autonomously stop the bleeding can be selected. As a result, improved safety in the medical operation can be achieved.

The determination unit 133 may determine whether external force applied to the medical operation manipulator 300 exceeds a predetermined threshold, and the selection unit 134 may select a switching sequence in accordance with whether the external force applied to the medical operation manipulator 300 exceeds the predetermined threshold.

With this configuration, an appropriate sequence can be selected in accordance with the magnitude of the external force applied to the medical operation manipulator 300 or the like. Accordingly, switching can be forcibly performed to the manual operation mode, for example, when the operation of the medical operation manipulator 300 driven in the autonomous drive mode is different from that expected by a doctor. As a result, the autonomous drive mode and the manual operation mode of the medical operation manipulator 300 can be securely switched.

The determination unit 133 may determine the status of the medical operation being performed in the medical operation room and change the threshold in accordance with the status of the medical operation.

With this configuration, the external force threshold based on which a switching sequence is changed can be switched in accordance with the status of the medical operation. Accordingly, switching can be forcibly performed from the autonomous drive mode to the manual operation mode by reducing the threshold when large force is not applied to the medical operation manipulator 300. As a result, the autonomous drive mode and the manual operation mode of the medical operation manipulator 300 can be securely switched.

The determination unit 133 may determine whether external force for correcting the operation of the medical operation manipulator 300 is applied, and the selection unit 134 may select a semi-autonomous drive mode for correcting the operation of the medical operation manipulator 300 when the determination unit 133 determines that external force for correcting the operation of the medical operation manipulator 300 in the autonomous drive mode is applied.

With this configuration, the operation of the medical operation manipulator 300 being autonomously driven can be manually corrected. Accordingly, a doctor can manually correct the operation of the medical operation manipulator 300 when the operation of the medical operation manipulator 300 being autonomously driven is different from that expected by the doctor. As a result, improved safety is achieved.

The control device 100 includes: the sensing unit 132 configured to sense a switching operation that performs switching between the autonomous drive mode and the manual operation mode of the medical operation manipulator 300; the determination unit 133 configured to determine the status of the medical operation room in which the medical operation manipulator 300 is disposed when the switching operation is sensed by the sensing unit 132; and the selection unit 134 configured to select a switching sequence in accordance with the status determined by the determination unit 133.

With this configuration, an appropriate sequence through which the autonomous drive mode and the manual operation mode are switched can be selected in accordance with the status of the medical operation room. As a result, the autonomous drive mode and the manual operation mode of the medical operation manipulator 300 can be securely switched.

A control method senses a switching operation that performs switching between the autonomous drive mode and the manual operation mode of the medical operation manipulator 300, determines the status of a medical operation room in which the medical operation manipulator 300 is disposed when the switching operation on the medical operation manipulator is sensed, and selects a switching sequence in accordance with the determined status.

With this method, an appropriate sequence through which the autonomous drive mode and the manual operation mode are switched can be selected in accordance with the status of the medical operation room. As a result, the autonomous drive mode and the manual operation mode of the medical operation manipulator 300 can be securely switched.

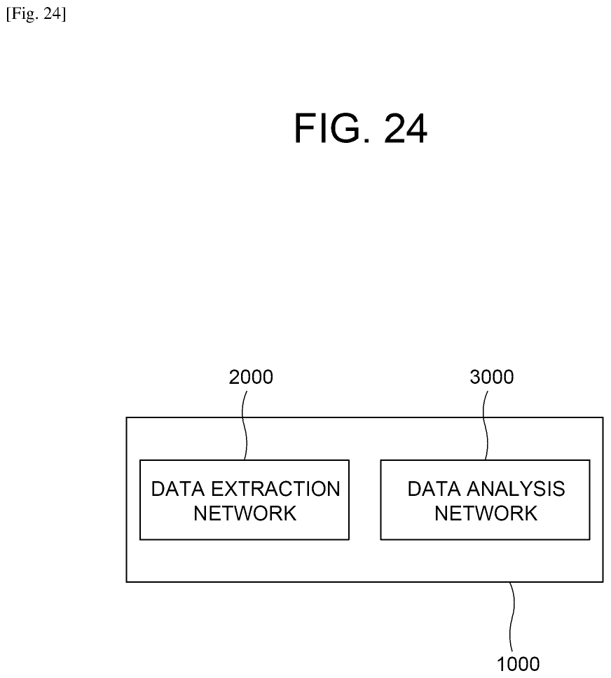

As will now be explained the computer 1000 may optionally use artificial intelligence (AI) to determine when to change between a more autonomous mode and a less autonomous mode, or vice versa. First, by referring to FIG. 24, a configuration of the computer 1000 will be explained.

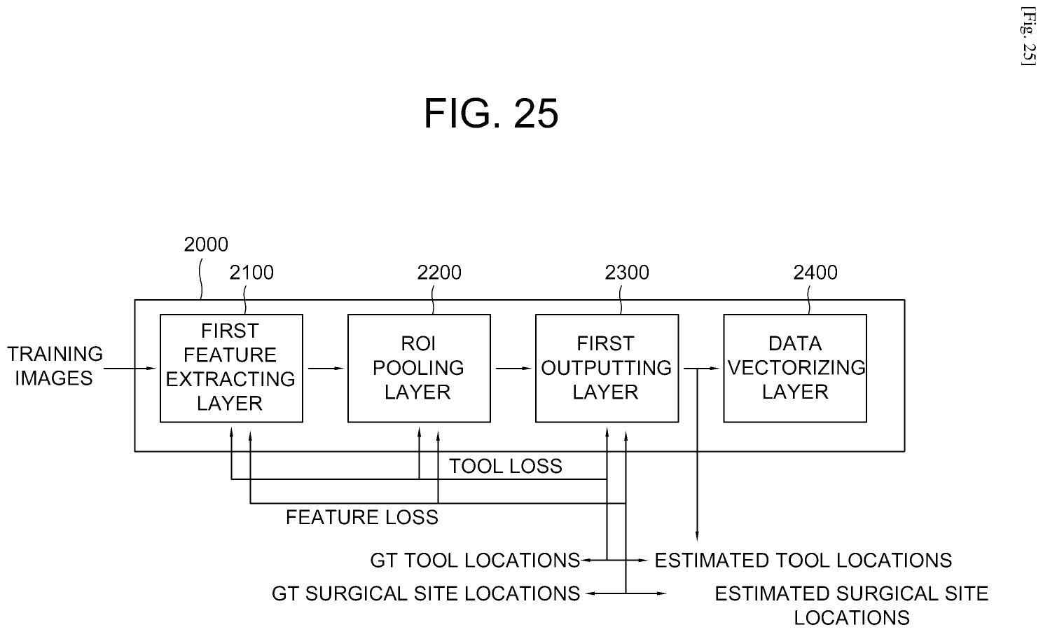

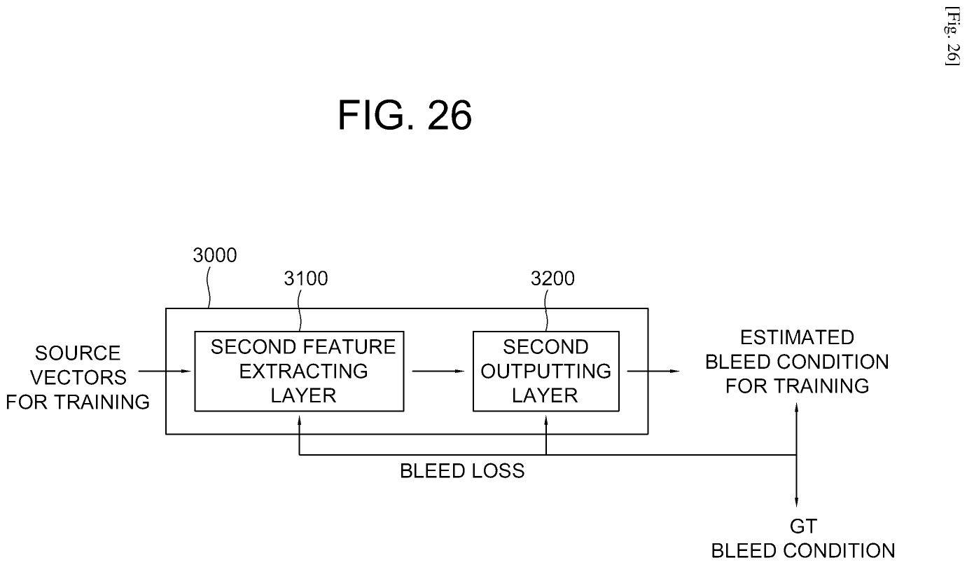

The computer 1000 may include a data extraction network 2000 and a data analysis network 3000. Further, as illustrated in FIG. 25, the data extraction network 2000 may include at least one first feature extracting layer 2100, at least one Region-Of-Interest (ROI) pooling layer 2200, at least one first outputting layer 2300 and at least one data vectorizing layer 2400. And, also to be illustrated in FIG. 26, the data analysis network 3000 may include at least one second feature extracting layer 3100 and at least one second outputting layer 3200.

Below, specific processes of calculating when to determine a mode shift will be presented.

First, to illustrate an example, suppose the computer 1000 acquires an image from a camera disposed on a distal end of the medical operation manipulator 300. The subject image may correspond to a scene of a surgery, photographed from a distal end of the front of the medical operation manipulator 300, including an image of a surgical scene that includes a surgical site, where there is at least some bleeding tissue and a portion of the subject's body that is adjacent to the surgical scene without bleeding tissue.

After the subject image (images and/or video) is acquired, in order to generate a source vector to be input to the data analysis network 3000, the computer 1000 may instruct the data extraction network 2000 to generate the source vector including (i) an apparent bleeding condition (degree of bleeding), which is present between a first part of the subject's body that does not include the surgical wound, and (ii) an apparent bleed amount at the surgical site, with a visible volume of exposed blood. The present example of an amount of bleeding is just an example of how the AI engine may be used to assist in mode control situations. It may be used in a similar manner for other situations, such as knowing whether to switch modes in scalpel operations, needle operations, generating a pseudo-weight, camera movement, manipulator movement, etc.

To generate the source vector, the computer 1000 may instruct at least part of the data extraction network 2000 to detect the surgical site and the unaffected portion of the subject's body relative to the surgical site on the subject image.

Specifically, the computer 1000 may instruct the first feature extracting layer 2100 to apply at least one first convolutional operation to the subject image, to thereby generate at least one subject feature map. Thereafter, the computer 1000 may instruct the ROI pooling layer 2200 to generate one or more ROI-Pooled feature maps by pooling regions on the subject feature map, corresponding to ROIs on the subject image which have been acquired from a Region Proposal Network (RPN) interworking with the data extraction network 2000. And, the computer 1000 may instruct the first outputting layer 2300 to generate at least one estimated unaffected (not bloody) portion of the body adjacent to the surgical wound and at least one estimated amount of blood at the surgical site. That is, the first outputting layer 2300 may perform a classification and a regression on the subject image, by applying at least one first Fully-Connected (FC) operation to the ROI-Pooled feature maps, to generate each of the estimated unaffected tissue location and the estimated portion of the wound site with exposed blood (not contained in a venous structure), including information on coordinates of each of bounding boxes. Herein, the bounding boxes may include the non-bloody site and the bloody site.

After such detecting processes are completed, by using the estimated non-bloody portion of the subject and the estimated blood amount in the surgical wound, the computer 1000 may instruct the data vectorizing layer 2400 to subtract a y-axis coordinate of an upper bound of the ground from a y-axis coordinate of the lower boundary of the bloody wound site to generate the apparent amount of exposed blood in the wound site, and multiply a vertical height of the wound site and a horizontal width of the wound site to generate an area in which an amount of exposed blood may be present in the surgery at the time the image was captured.

After the apparent height and the apparent size are acquired, the computer 1000 may instruct the data vectorizing layer 2400 to generate at least one source vector including the apparent height and the apparent size as its at least part of components.

Then, the computer 1000 may instruct the data analysis network 3000 to calculate an estimated amount of blood by using the source vector. Herein, the second feature extracting layer 3100 of the data analysis network 3000 may apply a second convolutional operation to the source vector to generate at least one source feature map, and the second outputting layer 3200 of the data analysis network 3000 may perform a regression, by applying at least one FC operation to the source feature map, to thereby calculate the estimated area of exposed tissue in which an amount of exposed blood may be present.

As shown above, the computer 1000 may include two neural networks, i.e., the data extraction network 2000 and the data analysis network 3000, which are examples of convolutional neural networks (CNNs). The two neural networks are trained to perform the processes properly. Below, how to train the two neural networks will be explained by referring to FIG. 25 and FIG. 26.

First, by referring to FIG. 25, the data extraction network 2000 may have been trained by using (i) a plurality of training images corresponding to scenes of subject surgeries for training, captured from a camera mounted to the medical operation manipulator for training, including images that include varying degrees of exposed blood for the kind of surgery, and area of wound, being performed and images of their corresponding non-bloody tissue sites adjacent to the surgical site for training, and (ii) a plurality of their corresponding GT non-bloody site locations and GT surgical sites. More specifically, the data extraction network 2000 may have applied aforementioned operations to the training images, and have generated their corresponding estimated non-bloody locations and estimated surgical site locations with varying degrees of exposed blood. Then, (i) each of non-bloody pairs of each of the estimated non-bloody locations and each of their corresponding GT non-bloody locations and (ii) each of surgical site pairs of each of the estimated surgical site locations and each of the GT surgical site locations may have been referred to, in order to generate at least one wound location loss and at least one exposed blood amount loss, by using any of loss generating algorithms, e.g., a smooth-L1 loss algorithm and a cross-entropy loss algorithm. Thereafter, by referring to the wound location loss and the blood amount loss, backpropagation may have been performed to learn at least part of parameters of the data extraction network 2000. In this process, the AI engine is trained based on the rules discussed above, such that weighting parameters are determined between nodes of the respective layers in the data extraction network 2000 for subsequent use when extracting features from images (non-training images) to be analyzed. Then the AI engine can be used for a particular surgery by applying a captured image of a surgical site to the AI engine so the exposed blood (as a feature) may be estimated, and based on that estimation (i.e., degree of exposed blood in the surgical site), the system may provide a refined degree on mode control. For example, for a higher than average amount of exposed blood, the system may choose to not switch from a less autonomous mode to a more autonomous mode because the greater degree of exposed blood may indicate that the surgery is riskier than a normal surgery. Other parameters of the RPN can be trained similarly.

The data vectorizing layer 2400 may have been implemented by using a rule-based algorithm, not a neural network algorithm. In this case, the data vectorizing layer 2400 may not need to be trained, and may just be able to perform properly by using its settings stored in advance. As an example, the first feature extracting layer 2100, the ROI pooling layer 2200 and the first outputting layer 2300 may be acquired by applying a transfer learning, as is known, to an existing object detection network such as VGG or ResNet, etc.

Second, by referring to FIG. 26, the data analysis network 3000 may have been trained by using (i) a plurality of source vectors for training, including apparent surgical sites for training and apparent sizes for training as their components, and (ii) a plurality of their corresponding GT bleed amounts. More specifically, the data analysis network 3000 may have applied aforementioned operations to the source vectors for training, to thereby calculate their corresponding estimated bleed amounts for training. Then each of bleed pairs of each of the estimated surgical sites and each of their corresponding GT bleed amounts may have been referred to, in order to generate at least one bleed loss, by using any of the loss algorithms. Thereafter, by referring to the bleed loss, backpropagation can be performed to learn at least part of parameters of the data analysis network 3000.

After performing such training processes, the computer 1000 can properly calculate the estimated bleed amounts by using the subject image including the scene photographed from the surgery.

Hereafter, another embodiment will be presented. In this embodiment, the source vector further includes a tilt angle, which is an angle between an optical axis of a camera which has been used for photographing the subject image (e.g., the surgical patient) and a vertical axis of the patient, as its additional component. Also, in order to calculate the tilt angle to be included in the source vector, the data extraction network of the second embodiment may be slightly different from that of the first one. In order to use the second embodiment, it should be assumed that information on a principal point and focal lengths of the camera are provided.

Specifically, in another embodiment, the data extraction network 2000 may have been trained to further detect certain types of tissue (e.g., an organ, or the kind of surgery such surgery on an artery) in the subject image, to thereby detect at least one vanishing point of the subject image. Herein, the tissue features may denote distinctive tissue representing boundaries of the surgical site in the subject image, and the vanishing point may denote where extended lines generated by extending the lines of boundaries are gathered. As an example, through processes performed by the first feature extracting layer 2100, the ROI pooling layer 2200 and the first outputting layer 2300, edges of surgical sites may be detected.

After the tissue features are detected, the data vectorizing layer 2400 may find at least one point where the most extended lines are gathered, and determine it as the vanishing point. Thereafter, the data vectorizing layer 2400 may calculate the tilt angle by referring to information on the vanishing point, the principal point and the focal lengths of the camera by using a following formula.

$${θ}_{tilt} = atan 2 ( v y - c y , f y ) \qquad [ Math .1 ]$$

In the formula, vy may denote a y-axis coordinate of the vanishing point, cy may denote a y-axis coordinate of the principal point, and fy may denote a y-axis focal length.

After the tilt angle is calculated, the data vectorizing layer 2400 may set the tilt angle as a component of the source vector, and the data analysis network 3000 may use such source vector to calculate the estimated extent of the wound and the amount of bleeding. In this case, the data analysis network 3000 may have been trained by using the source vectors for training additionally including tilt angles for training.

As another embodiment, the source vector may further include an actual distance, which is a distance in a real world between the camera and the surgical site, as an additional component of the source vector. For this embodiment, it is assumed that a camera height, which is a distance between the camera and a reference (e.g., surgical table) directly below the camera in the real world, is provided. This embodiment is the same as the embodiment discussed above until the first outputting layer 2300 generates the tilt angle. Hereinafter, processes performed after the tilt angle is generated will be explained.

The computer 1000 may instruct the data analysis network 3000 to calculate the actual distance by referring to information on the camera height, the tilt angle, a coordinate of the lower boundary of the surgical wound, by using a following formula.

$${d}_{actual} = \sqrt{\frac{{h}^{2} + {h}^{2} {tan}^{2} \{ \frac{π}{2} + {θ}_{tilt} - atan ( \frac{y - cy}{f y} ) \}}{1 + \frac{{( y - cy )}^{2}}{f {y}^{2}}} {( \frac{x - c x}{f x} )}^{2} + \\ {h}^{2} {tan}^{2} \{ \frac{π}{2} + {θ}_{tilt} - atan ( \frac{y - c y}{f y} ) \}} \qquad [ Math .2 ]$$

In the formula, x and y may denote coordinates of the lower boundary of the surgical site, fx and fy may denote the focal lengths for each axis, cx and cy may denote coordinates of the principal point, and h may denote the camera height. A usage of such formula for calculating the actual distance is known, thus further explanation is omitted. Alternatively, or complementarily, the system also uses time of flight (ToF) sensor to measure distance.

After the actual distance is calculated, the data vectorizing layer 2400 may set the actual distance as the additional component of the source vector, and the data analysis network 3000 may use such source vector to calculate the estimated size of the wound, and associated amount of blood. Also, in this case, the data analysis network 3000 may have been trained by using the source vectors for training additionally including actual distances for training.

For another embodiment which is mostly similar to the first one, some information acquired from a subject surgical database storing information on subject surgical procedures, including the subject surgical procedure, can be used for generating the source vector. That is, the computer 1000 may acquire structure information on a characteristic (e.g., size of wound, location of surgery, organs exposed, as well as normal and abnormal amounts of bleeding of the subject surgery etc.) from a database. Herein, at least one of the characteristics information can be added to the source vector by the data vectorizing layer 2400, and the data analysis network 3000, which has been trained by using the source vectors for training additionally including corresponding information, i.e., at least one of the characteristics information, may use such source vector to calculate the estimated bleed amount, which in turn is used to trigger a change in control mode based on a threshold comparison of the detected blood versus an expected blood for that kind of surgical condition.

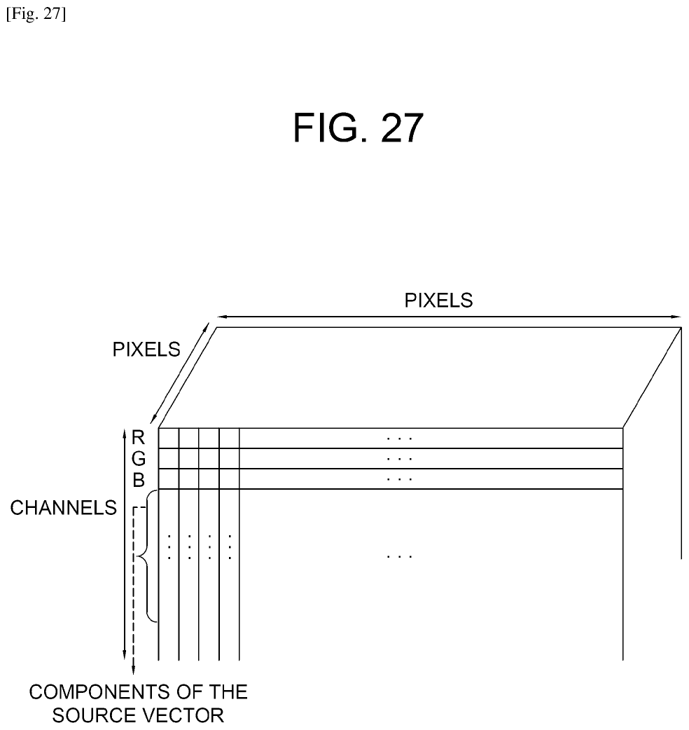

As another embodiment, the source vector, generated by using any of the earlier embodiments, can be concatenated channel-wise to the subject image or its corresponding subject segmented feature map, which has been generated by applying an image segmentation operation thereto, to thereby generate a concatenated source feature map, and the data analysis network 3000 may use the concatenated source feature map to calculate the estimated blood amount. An example configuration of the concatenated source feature map may be shown in FIG. 27. In this case, the data analysis network 3000 may have been trained by using a plurality of concatenated source feature maps for training including the source vectors for training, other than using only the source vectors for training. By using the present embodiment, much more information can be inputted to processes of calculating the estimated exposed blood amount, thus it can be more accurate. Herein, if the subject image is used directly for generating the concatenated source feature map, it may require too much computing resources, thus the subject segmented feature map may be used for reducing a usage of the computing resources.

Descriptions above are explained under an assumption that the subject image has been photographed from the front-top of the subject. However, embodiments stated above may be adjusted to be applied to the subject image photographed from other sides of the subject.

The effects described in the present specification are merely exemplary and not restrictive, and may include any other effect.

The present technology may have configurations as follows.

(1)
According to one embodiment, a medical tool control system includes a medical operation manipulator that detachably holds a medical tool; and
circuitry configured to
   receive an input signal from an external device,
   evaluate a content of the input signal so as to determine a change in operation mode from a first control mode to a second control mode of the medical operation manipulator, wherein the second control mode has greater degree of autonomy than the first control mode, and
in the second control mode, generate a control signal to drive a movement of the medical operation manipulator.

(2)
According to one aspect, the medical tool control system of (1), further includes a controllable actuator that drives a movement of at least a part of the medical operation manipulator based on the control signal from the circuitry.

(3)
According to another aspect, the medical tool control system of (1) or (2), wherein the external device comprises a sensor, the sensor including at least one of an image sensor, an origin sensor, a motion sensor, a microphone, an acceleration sensor, a force sensor, and a pressure sensor.

(4)
The medical tool control system of any one of (1) to (3), wherein
the input signal includes information regarding a vicinity of the medical tool relative to a predetermined position, and
the circuitry is configured to determine the change in operation mode based on the vicinity of the medical tool relative to the predetermined position.

(5)
The medical tool control system of any one of (1) to (4), wherein the circuitry is configured to determine the change in operation mode based on whether the medical operation manipulator is already in the second mode of operation, the second mode of operation being an autonomous mode of operation.

(6)
The medical tool control system of any one of (1) to (5), wherein the circuitry is configured to determine the change in operation mode in accordance with which medical tool is attached to the medical operation manipulator.

(7)
The medical tool control system of (6), wherein
the medical operation manipulator is configured to hold a camera, and
the circuitry is further configured to switch from the second control mode to the first control mode in response to an external force being applied to at least one of the camera or the medical operation manipulator for a predetermined duration.

(8)
The medical tool control system of (6), wherein
the medical operation manipulator is configured to hold a scalpel, and
the circuitry is further configured to switch from the second control mode to the first control mode in response to an external force being applied to at least one of the scalpel or the medical operation manipulator for a predetermined duration.

(9)
The medical tool control system of (6), wherein
the medical operation manipulator is configured to hold a needle, and
the circuitry is further configured to switch from the second control mode to the first control mode in response to at least one of
an external force being applied to at least one of the needle or the medical operation manipulator for a predetermined duration, or
the needle being inserted in a subject or removed from the subject.

(10)
The medical tool control system of (6), wherein
the medical operation manipulator is configured hold a retractor, and
the circuitry is configured to control the medical operation manipulator to produce a pseudo weight in response to a determination that the retractor is holding an organ.

(11)
The medical tool control system of any one of (1) to (10), wherein the medical operation manipulator including an articulated arm.

(12)
The medical tool control system of any one of (1) to (11), wherein the circuitry is further configured to determine the change in operation mode according to whether an attendant to the medical operation manipulator is a medical doctor, the input signal including information about the attendant.

(13)
The medical tool control system of any one of (1) to (12), wherein the circuitry is further configured to determine the change in operation mode according to a status of a surgery being performed.

(14)
The medical tool control system of (13), wherein the status of the surgery being performed includes a degree of progress of the surgery.

(15)
The medical tool control system of (13), wherein the circuitry is configured to change a time at which the operation mode is changed to the second control mode, or changed from the second control mode back to the first control mode, in accordance with the degree of the progress.

(16)
The medical tool control system of (13), wherein the circuitry is configured to change a time at which the operation mode is changed to the second control mode, or changed from the second control mode back to the first control mode, in accordance with a detected amount of bleeding at a surgical site.

(17)
The medical tool control system of any one of (1) to (16), wherein
the circuitry is further configured to switch operation modes in response to an external force applied to the medical operation manipulator or the medical tool that exceeds a predetermined threshold.

(18)
The medical tool control system of (17), wherein the predetermined threshold is changed in accordance with a status of a surgery.

(19)
The medical tool control system of any one of (1) to (18), wherein the circuitry is configured to switch back to the first control mode to modify a movement of the medical operation manipulator in response to an external force applied to the medical operation manipulator or the medical tool to modify a movement of the medical operation manipulator in the second control mode.

(20)
The medical tool control system of any one of (1) to (19), wherein the circuitry is configured to implement the change in operation mode as part of a transition process that occurs over a predetermined period of time.

(21)
The medical tool control system of (1), wherein the second control mode has a greater degree of autonomy than the first control mode.

(22)
The medical tool control system of (1), wherein the first control mode has a greater degree of autonomy than the second control mode.

(23)
According to a second embodiment, a controller for a medical operation manipulator that detachable holds a medical tool is described, the controller including:
circuitry configured to
   receive an input signal from an external device,
   evaluate a content of the input signal to determine a change in operation mode from a first control mode to a second control mode of the medical operation manipulator, wherein the second control mode has greater degree of autonomy than the first control mode, and
   in the second control mode, generate a control signal to drive a movement of the medical operation manipulator.

(24)
According to a third embodiment, a non-transitory computer readable storage is described that has instructions that when executed by a processor cause the processor to perform a method, the method including:
receiving an input signal from an external device;
evaluating with circuitry a content of the input signal and determining a change in operation mode from a first control mode to a second control mode of a medical operation manipulator, wherein the second control mode has greater degree of autonomy than the first control mode; and
in the second control mode, generating a control signal to drive a movement of the medical operation manipulator.

Alternatively, the present technology may have configurations as follows.

(1)
A medical operation support system including:
a medical operation manipulator;
a sensing unit configured to sense a switching operation that performs switching between an autonomous drive mode and a manual operation mode of the medical operation manipulator;
a determination unit configured to determine the status of a medical operation room in which the medical operation manipulator is disposed when the switching operation is sensed by the sensing unit; and
a selection unit configured to select a switching sequence in accordance with the status determined by the determination unit.

(2)
The medical operation support system according to (1), wherein
the determination unit determines the state of the medical operation manipulator, and
the selection unit selects a switching sequence in accordance with the state of the medical operation manipulator.

(3)
The medical operation support system according to (1) or (2), wherein the determination unit determines whether the medical operation manipulator is in the autonomous drive mode or the manual operation mode.

(4)
The medical operation support system according to (1) or (2), wherein
the determination unit determines a medical instrument mounted on the medical operation manipulator, and
the selection unit selects a switching sequence in accordance with the medical instrument mounted on the medical operation manipulator.

(5)
The medical operation support system according to (4), wherein the determination unit determines whether the medical operation manipulator is in the autonomous drive mode or the manual operation mode, and
the selection unit selects a switching sequence in accordance with a medical instrument mounted on the medical operation manipulator and whether the medical operation manipulator is in the autonomous drive mode or the manual operation mode.

(6)
The medical operation support system according to (5), wherein
when it is determined that the medical instrument is a camera or a scalpel and when the medical operation manipulator is in the autonomous drive mode,
the selection unit selects a switching sequence through which switching is performed to the manual operation mode in stages.

(7)
The medical operation support system according to (5), wherein
when it is determined that the medical instrument is a needle holder and when the medical operation manipulator is in the autonomous drive mode and a needle grasped by the needle holder is inserted into an affected part,
the selection unit selects a switching sequence through which switching is performed to the manual operation mode in stages.

(8)
The medical operation support system according to (5), wherein
when it is determined that the medical instrument is a retractor and when the medical operation manipulator is in the autonomous drive mode and the retractor holds an organ,
the selection unit selects a switching sequence through which pseudo weight is generated at the medical operation manipulator.

(9)
The medical operation support system according to (8), wherein
the selection unit selects a switching sequence through which switching is performed to the manual operation mode upon reception of predetermined operation after pseudo weight is generated at the medical operation manipulator.

(10)
The medical operation support system according to any one of (1) to (9), wherein the medical operation manipulator includes an arm unit at least partially bendable and capable of supporting the medical instrument.

(11)
The medical operation support system according to any one of (1) to (10), wherein
the determination unit determines the state of an operator having executed the switching operation on the medical operation manipulator, and
the selection unit selects a switching sequence in accordance with the state of the operator.

(12)
The medical operation support system according to (11), wherein
the determination unit determines the attribute of the operator, and
the selection unit selects a switching sequence in accordance with the attribute of the operator.

(13)
The medical operation support system according to (11) or (12), wherein
the determination unit determines whether the operator is a doctor, and
the selection unit selects a switching sequence in accordance with whether the operator is a doctor.

(14)
The medical operation support system according to any one of (11) to (13), wherein
the determination unit determines a record of biological information of the operator, and
the selection unit selects a switching sequence in accordance with the record of biological information of the operator.

(15)
The medical operation support system according to any one of (1) to (14), wherein
the determination unit determines the status of a medical operation being performed in the medical operation room, and
the selection unit selects a switching sequence in accordance with the status of the medical operation being performed in the medical operation room.

(16)
The medical operation support system according to any one of (1) to (15), wherein
the determination unit determines the degree of progress of the medical operation being performed in the medical operation room, and
the selection unit selects a switching sequence in accordance with the degree of progress of the medical operation being performed in the medical operation room.

(17)
The medical operation support system according to (16), wherein the selection unit selects a switching sequence having a different transition time in accordance with the degree of progress of the medical operation being performed in the medical operation room.

(18)
The medical operation support system according to any one of (1) to (17), wherein
the determination unit determines existence of bleeding in an operative field, and
the selection unit selects a switching sequence in accordance with the existence of bleeding in the operative field.

(19)
The medical operation support system according to any one of (1) to (18), wherein
the determination unit determines whether a doctor is present in the vicinity of the medical operation manipulator, and
the selection unit selects a switching sequence in accordance with whether a doctor is present in the vicinity of the medical operation manipulator.

(20)
The medical operation support system according to (19), wherein the selection unit selects a switching sequence through which the medical operation manipulator is caused to stop bleeding in the operative field when it is determined that no doctor is present in the vicinity of the medical operation manipulator and the medical operation manipulator is in the autonomous drive mode.

(21)
The medical operation support system according to any one of (1) to (20), wherein the determination unit determines whether external force applied to the medical operation manipulator exceeds a predetermined threshold, and the selection unit selects a switching sequence in accordance with whether the external force applied to the medical operation manipulator exceeds the predetermined threshold.

(22)
The medical operation support system according to (21), wherein the determination unit determines the status of a medical operation being performed in the medical operation room and changes the threshold in accordance with the status of the medical operation.

(23)
The medical operation support system according to any one of (1) to (22), wherein
the determination unit determines whether external force for correcting operation of the medical operation manipulator is applied, and
the selection unit selects a semi-autonomous drive mode for correcting operation of the medical operation manipulator when the determination unit determines that external force for correcting operation of the medical operation manipulator in the autonomous drive mode is applied.

(24)
A control device including:
a sensing unit configured to sense a switching operation that performs switching between an autonomous drive mode and a manual operation mode on a medical operation manipulator;
a determination unit configured to determine the status of a medical operation room in which the medical operation manipulator is disposed when the switching operation is sensed by the sensing unit; and
a selection unit configured to select a switching sequence in accordance with the status determined by the determination unit.

(25)
A control method including:
sensing a switching operation that performs switching between an autonomous drive mode and a manual operation mode on a medical operation manipulator;
determining the status of a medical operation room in which the medical operation manipulator is disposed when the switching operation on the medical operation manipulator is sensed; and
selecting a switching sequence in accordance with the determined status.

### REFERENCE SIGNS LIST

1 Medical operation support system
100 Control device
110 Communication unit
120 Storage unit
130 Control unit
131 Acquisition unit
132 Sensing unit
133 Determination unit
134 Selection unit
135 Manipulator control unit
136 Communication control unit
1000 Computer
2000 Data extraction network
2100 First feature extracting layer
2200 Region of interest pooling layer
2300 First outputting layer
2400 Data vectorizing layer
3000 Data analysis network
3100 Second feature extracting layer
3200 Second outputting layer

1. A medical tool control system comprising:
a medical operation manipulator that detachably holds a medical tool;
and
circuitry configured to
receive an input signal from an external device,
evaluate a content of the input signal so as to determine a change in operation mode from a first control mode to a second control mode of the medical operation manipulator, wherein the first control mode and the second control mode have differing degrees of autonomy, and
in at least one of the first control mode and the second control mode, the circuitry generates a control signal to drive a movement of the medical operation manipulator.

2. The medical tool control system of claim 1, further comprising:
a controllable actuator that drives a movement of at least a part of the medical operation manipulator based on the control signal from the circuitry.

3. The medical tool control system of claim 1, wherein the external device comprises a sensor, the sensor including at least one of an image sensor, a location sensor, an encoder, a motion sensor, a microphone, an acceleration sensor, a force sensor, or a pressure sensor.

4. The medical tool control system of claim 1, wherein
the input signal includes information regarding a vicinity of the medical tool relative to a predetermined position, and
the circuitry is configured to determine the change in operation mode based on the vicinity of the medical tool relative to the predetermined position.

5. The medical tool control system of claim 1, wherein the circuitry is configured to determine the change in operation mode to the first control mode based on whether the medical operation manipulator is already in the second mode of operation, the second control mode being an autonomous mode of operation.

6. The medical tool control system of claim 1, wherein the circuitry is configured to determine the change in operation mode in accordance with which medical tool is attached to the medical operation manipulator.

7. The medical tool control system of claim 6, wherein
the medical operation manipulator is configured to hold a camera, and
the circuitry is further configured to switch from the second control mode to the first control mode in response to an external force being applied to at least one of the camera or the medical operation manipulator for a predetermined duration, the second control mode having a greater degree of autonomy than the first control mode.

8. The medical tool control system of claim 6, wherein
the medical operation manipulator is configured to hold a scalpel, and
the circuitry is further configured to switch from the second control mode to the first control mode in response to an external force being applied to at least one of the scalpel or the medical operation manipulator for a predetermined duration, the second control mode having a greater degree of autonomy than the first control mode.

9. The medical tool control system of claim 6, wherein
the medical operation manipulator is configured to hold a needle, and
the circuitry is further configured to switch from the second control mode to the first control mode in response to at least one of
an external force being applied to at least one of the needle or the medical operation manipulator for a predetermined duration, or
the needle being inserted in a subject or removed from the subject, the second control mode having a greater degree of autonomy than the first control mode.

10. The medical tool control system of claim 6, wherein
the medical operation manipulator is configured hold a retractor, and
the circuitry is configured to control the medical operation manipulator to produce a pseudo weight.

11. The medical tool control system of claim 6, wherein the medical operation manipulator comprising an articulated arm.

12. The medical tool control system of claim 1, wherein the circuitry is further configured to determine the change in operation mode according to whether an attendant to the medical operation manipulator is a medical doctor, the input signal including information about the attendant.

13. The medical tool control system of claim 1, wherein the circuitry is further configured to determine the change in operation mode according to a status of a surgery being performed.

14. The medical tool control system of claim 13, wherein the status of the surgery being performed includes a degree of progress of the surgery.

15. The medical tool control system of claim 13, wherein the circuitry is configured to change a time at which the operation mode is changed to the second control mode, or changed from the second control mode back to the first control mode, in accordance with the degree of the progress, the second control mode having a greater degree of autonomy than the first control mode.

16. The medical tool control system of claim 13, wherein the circuitry is configured to change a time at which the operation mode is changed in accordance with a detected amount of bleeding at a surgical site.

17. The medical tool control system of claim 1, wherein
the circuitry is further configured to switch operation modes in response to an external force applied to the medical operation manipulator or the medical tool that exceeds a predetermined threshold.

18. The medical tool control system of claim 17, wherein
the predetermined threshold is changed in accordance with a status of a surgery.

19. The medical tool control system of claim 1, wherein the circuitry is configured to switch to the first control mode to modify a movement of the medical operation manipulator in response to an external force applied to the medical operation manipulator or the medical tool to modify a movement of the medical operation manipulator in the second control mode, the second control mode having a greater degree of autonomy than the first control mode.

20. The medical tool control system of claim 1, wherein
the circuitry is configured to implement the change in operation mode as part of a transition process that occurs over a predetermined period of time.

21. The medical tool control system of claim 1, wherein
the second control mode has a greater degree of autonomy than the first control mode.

22. The medical tool control system of claim 1, wherein
the first control mode has a greater degree of autonomy than the second control mode.

23. A controller for a medical operation manipulator that detachable holds a medical tool, the controller comprising:
circuitry configured to
receive an input signal from an external device,
evaluate a content of the input signal to determine a change in operation mode from a first control mode to a second control mode of the medical operation manipulator, wherein the first control mode and the second control mode have differing degrees of autonomy, and
in at least one of the first control mode and the second control mode, the circuitry generates a control signal to drive a movement of the medical operation manipulator.

24. A non-transitory computer readable storage having instructions that when executed by a processor cause the processor to perform a method, the method comprising:
receiving an input signal from an external device;
evaluating with circuitry a content of the input signal and determining a change in operation mode from a first control mode to a second control mode of a medical operation manipulator, wherein the first control mode and the second control mode have differing degrees of autonomy, and
in at least one of the first control mode and the second control mode, the circuitry generates a control signal to drive a movement of the medical operation manipulator.
